(19) United States
(12) Patent Application Publication
(10) Pub. No.: US 2025/0186449 A1
(43) Pub. Date: Jun. 12, 2025

(54) COMPOSITIONS AND METHODS FOR TREATING A V-ATPASE MALFUNCTION

(71) Applicant: Kammy Moalemzadeh, New York, NY (US)

(72) Inventor: Kammy Moalemzadeh, New York, NY (US)

(21) Appl. No.: 19/061,728

(22) Filed: Feb. 24, 2025

Related U.S. Application Data
(63) Continuation-in-part of application No. PCT/US23/30793, filed on Aug. 22, 2023.
(60) Provisional application No. 63/423,854, filed on Nov. 9, 2022.
(60) Provisional application No. 63/413,424, filed on Oct. 5, 2022.
(60) Provisional application No. 63/399,964, filed on Aug. 22, 2022.

(51) Int. Cl. A61K 31/519; A61K 31/7008

(52) U.S. Cl. CPC A61K 31/519; A61K 31/7008

(57) ABSTRACT

A method for treating a V-ATPase malfunction may include identifying a malfunction of at least apportion of a V-ATPase protein, where the malfunction occurs in a transmembrane portion of the V-ATPase protein. Further, the method may include preparing a glycosylation precursor and preparing a P2Y12 inhibitor. Moreover, the method may include combining the glycosylation precursor and the P2Y12 inhibitor to yield a novel cocktail, and delivering, using a prodrug delivery system, the novel cocktail, wherein the prodrug delivery system comprises a prodrug.

### CROSS-REFERENCE TO RELATED APPLICATIONS

This application is a continuation in part of Patent Cooperation Treaty Application No. PCT/US23/30793 filed on Aug. 22, 2023 and entitled “COMPOSITIONS AND METHODS FOR TREATING A V-ATPASE MALFUNCTION” which claims the benefit of priority of U.S. Provisional Patent Application Ser. No. 63/399,964, filed on Aug. 22, 2022, and titled “A COMPOSITION FOR THE TREATMENT OF AN ATP6AP2 DEFICIENCY,” U.S. Provisional Patent Application Ser. No. 63/423,854, filed on Nov. 9, 2022, and titled “A METHOD, COMPOSITION, AND SYSTEM FOR TREATING A V-ATPASE MALFUNCTION,” and U.S. Provisional Patent Application Ser. No. 63/413,424, filed on Oct. 5, 2022, and titled “COMPOSITION FOR THE TREATMENT OF AN ATP6AP2 DEFICIENCY,” each of which are incorporated by reference herein in their entirety.

### FIELD OF THE INVENTION

The present invention generally relates to the field of therapeutics. In particular, the present invention is directed to a method, composition and system for treating a V-ATPase malfunction.

### BACKGROUND

V-ATPase is a protein complex that serves as a proton pump on cell and organelle membranes. Some individuals have malfunctioning V-ATPase due to, for example, a mutation that causes a subunit or other associated protein to not be produced in sufficient quantities or not to fold correctly. Defects in V-ATPase are apparent in various diseases and disorders including but not limited to Parkinson's, distal renal acidosis, myopathy, lysosomal storage disorders, congenital disorders of glycosylation, and the like. Defects in V-ATPase may occur at one or more locations of a V-ATPase protein. For example, ATP6AP2 is an assembly factor that mediates the formation of the multi-unit V-ATPase. ATP6AP2 may interact with ATP6AP1 to bind and chaperone a connection between subunits of a proton pump. Defects in ATP6AP2 may cause diseases associated with insufficient functional V-ATPase. It may be advantageous and beneficial to ensure that V-ATPase and ATP6AP2 function properly in efforts to remedy any possible defects and/or diseases.

### SUMMARY OF THE DISCLOSURE

In an aspect, a method of treating one or more V-ATPase malfunctions includes identifying a malfunction of at least a portion of a V-ATPase protein in a subject, manufacturing a composition comprising a glycosylation precursor, a modulator, and a delivery vehicle, and administering the composition to the subject.

In another aspect, a pharmaceutical composition may include a glycosylation precursor, a modulator, and a delivery vehicle.

These and other aspects and features of non-limiting embodiments of the present invention will become apparent to those skilled in the art upon review of the following description of specific non-limiting embodiments of the invention in conjunction with the accompanying drawings. BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

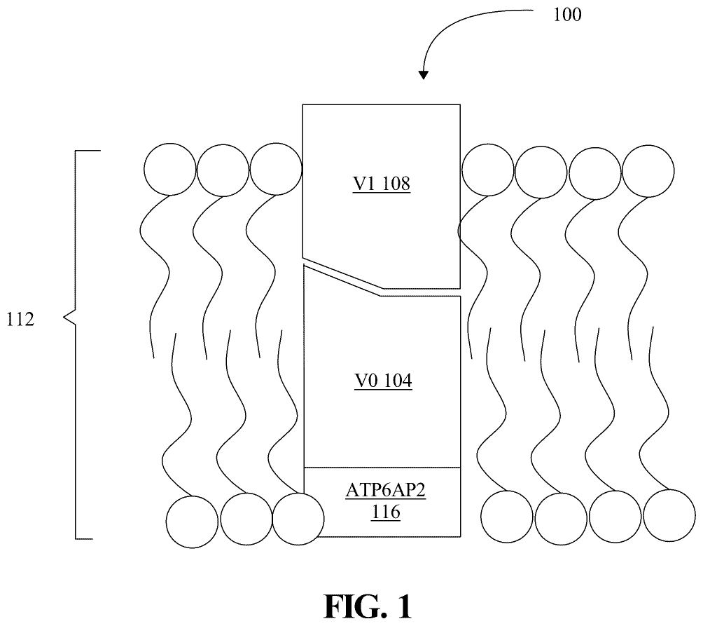

FIG. 1 illustrates an exemplary embodiment of a diagram of a V-ATPase protein according to an embodiment of the invention;

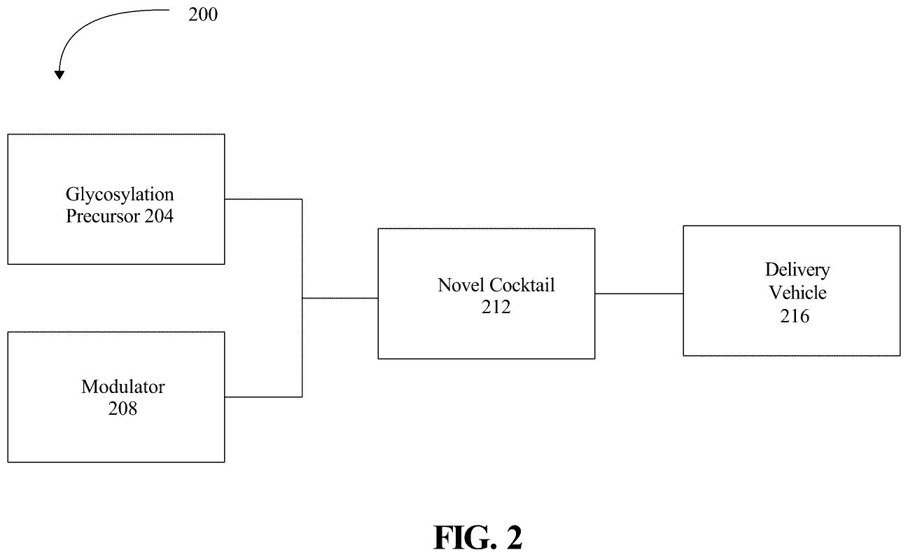

FIG. 2 illustrates an exemplary embodiment of a block diagram of an exemplary composition for treating a V-ATPase malfunction;

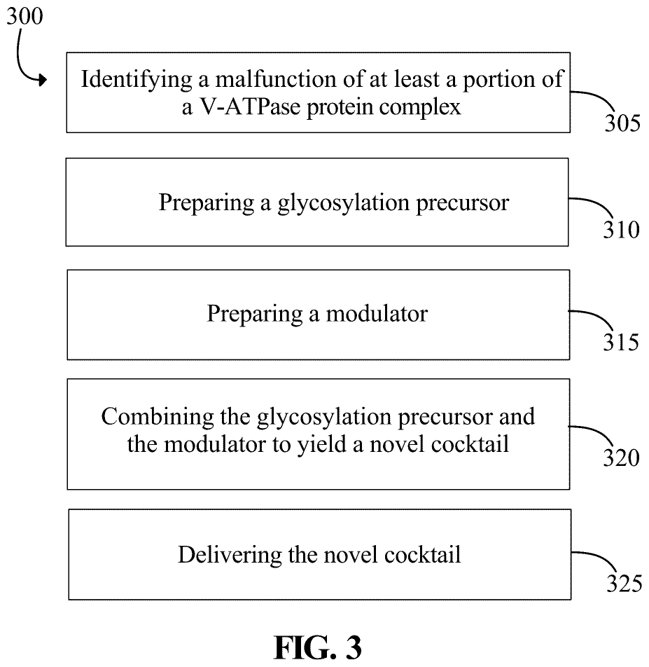

FIG. 3 illustrates an exemplary embodiment of a flow diagram of an exemplary method for treating a V-ATPase protein malfunction according to an embodiment of the invention;

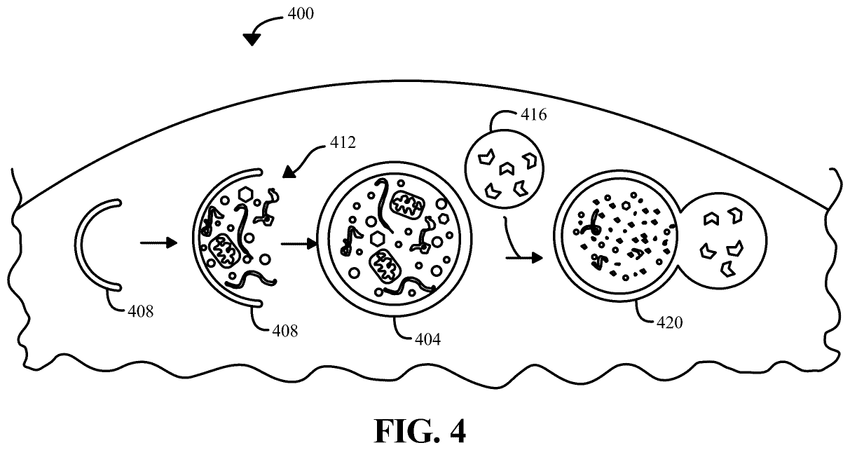

FIG. 4 illustrates an exemplary embodiment of an illustrative example of a lysosomal process;

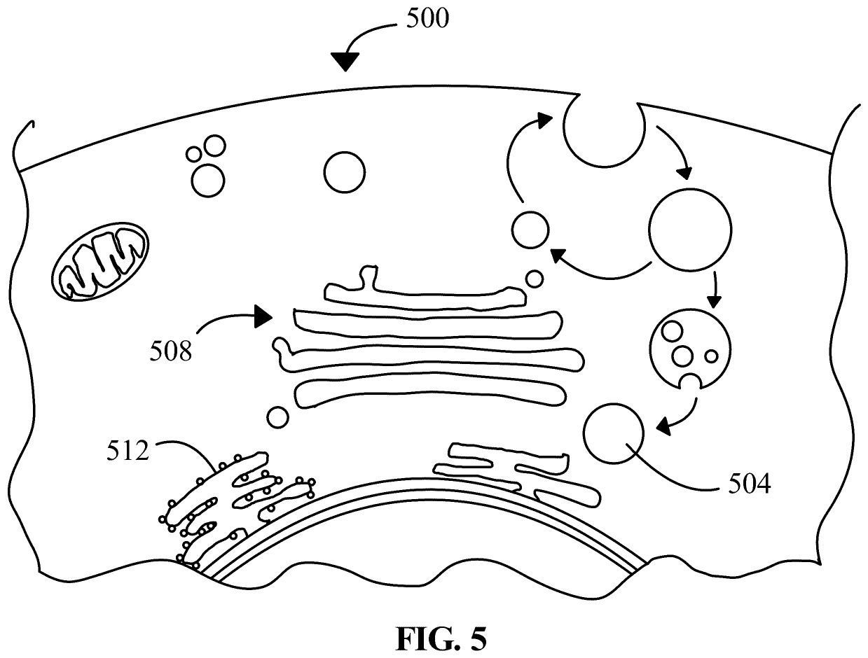

FIG. 5 illustrates an exemplary embodiment of an exemplary view of pH environments within a cell;

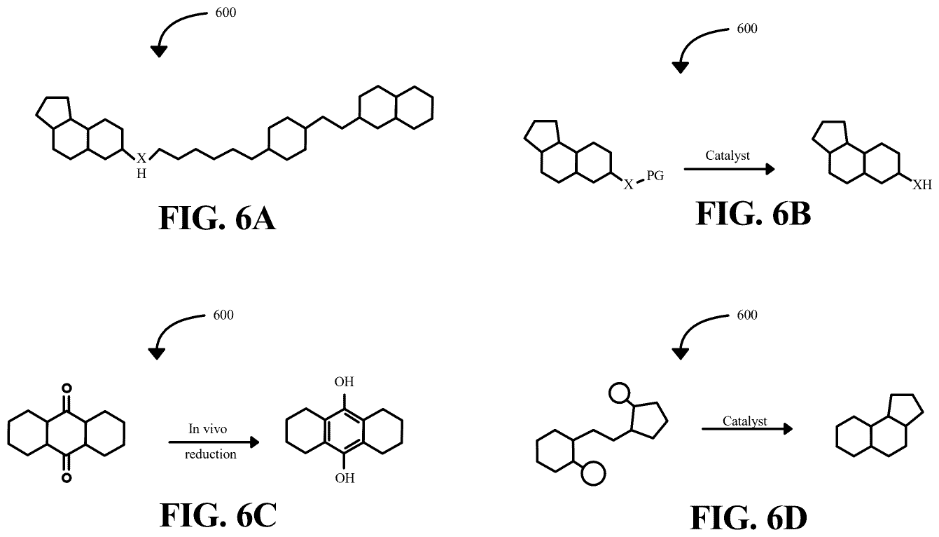

FIGS. 6A-D illustrate exemplary embodiments of prodrug structure;

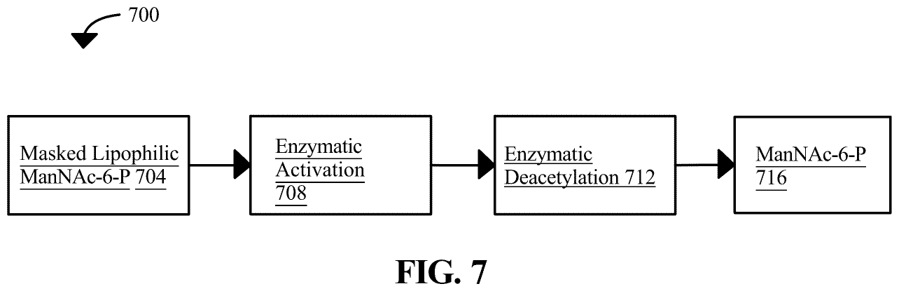

FIG. 7 illustrates an exemplary embodiment of a diagram of an exemplary prodrug delivery system with ManNAc;

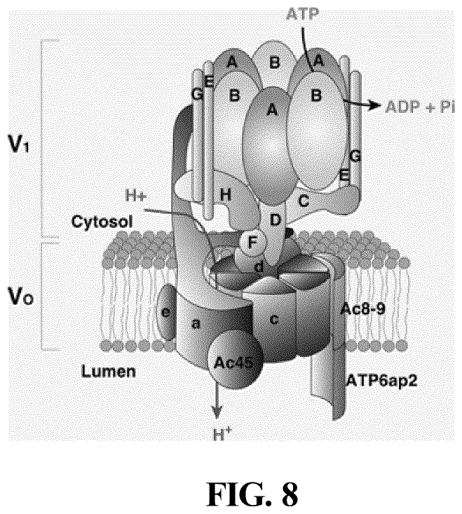

FIG. 8 illustrates an exemplary embodiment of a V-ATPase with ATP6ap2 subunit and V1 and V2 regions labeled;

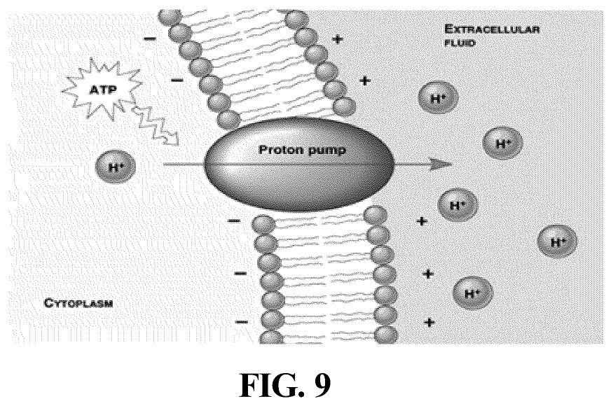

FIG. 9 illustrates an exemplary embodiment of a proton pump generating acidic environments across a membrane gradient;

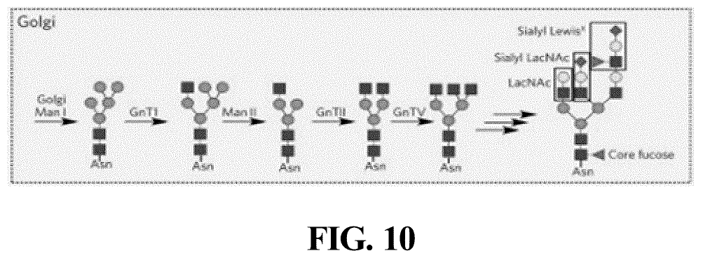

FIG. 10 illustrates an exemplary embodiment of addition of sialic acid to synthesized proteins;

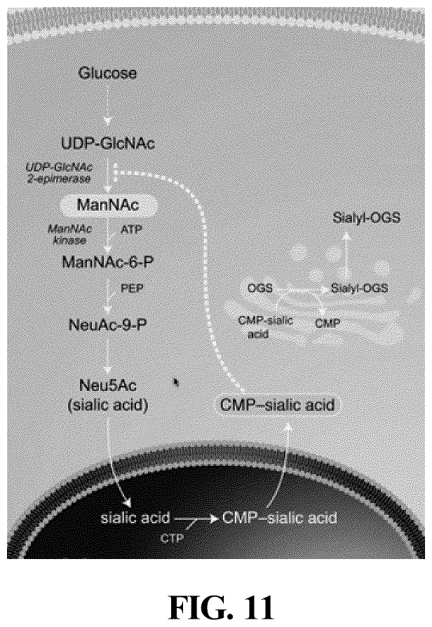

FIG. 11 illustrates an exemplary embodiment of glycosylation precursors and chemical reactions that convert them into sialic acid;

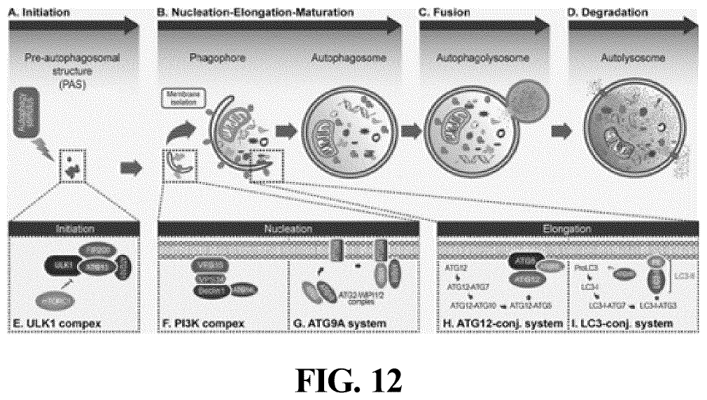

FIG. 12 illustrates an exemplary embodiment of the Autophagy-Lysosomal Pathway (ALP) as it relates to lysosomal fusion and degradation of waste;

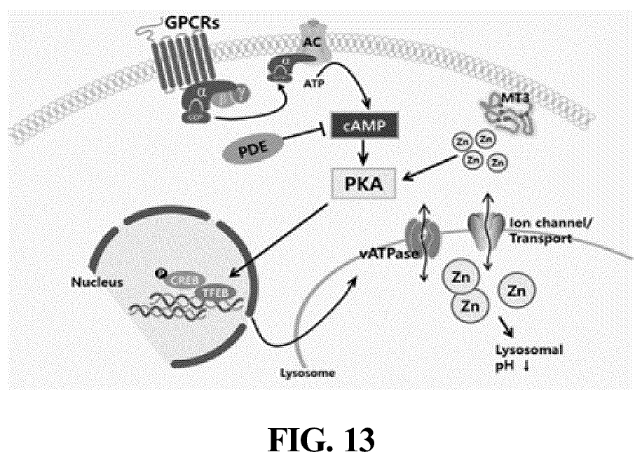

FIG. 13 illustrates an exemplary embodiment of TFEB, AMPK, and V-ATPase interaction as it relates to lysosome function and biogenesis;

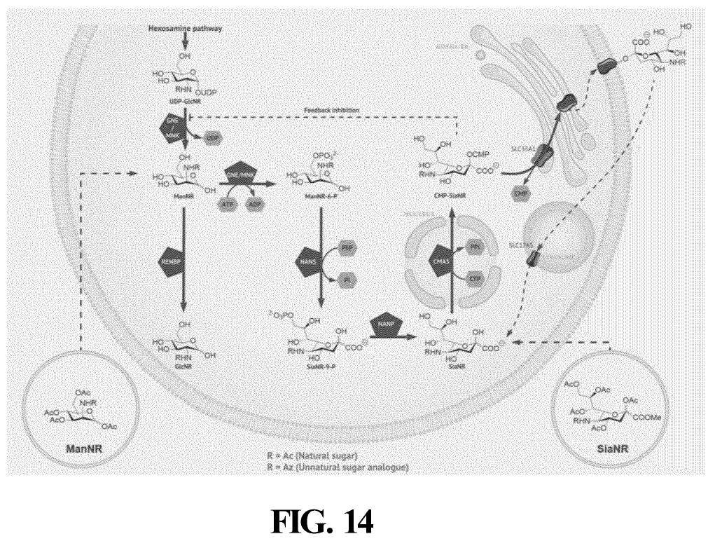

FIG. 14 illustrates a glycosylation therapeutic pathway;

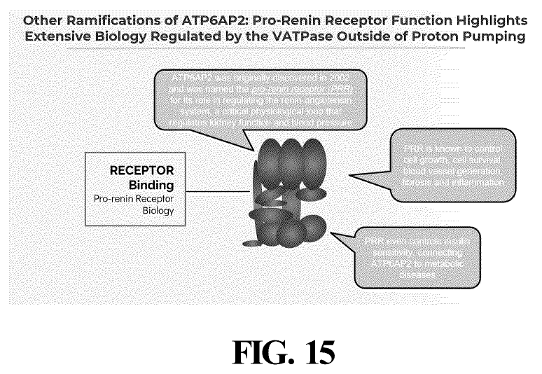

FIG. 15 illustrates metabolic effects of ATP6AP2; and

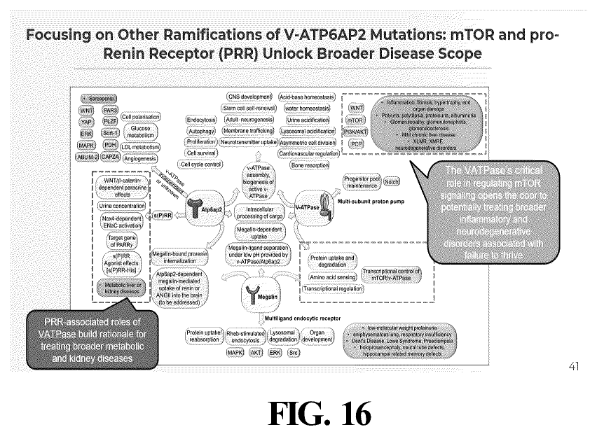

FIG. 16 illustrates V-ATPase malfunctions and mechanistic target of rapamycin (mTOR) signaling.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for treating a V-ATPase malfunction.

Aspects of the present disclosure may be used to treat a patient suffering from a mutation in V-ATPase that causes it to lose function or have decreased function, such as in a case where V-ATPase fails to properly assemble. Decreased or lost V-ATPase function may cause the pH of a cell and/or an organelle to differ from its physiologically appropriate levels. This may cause a variety of problems, including poor glycosylation of proteins and disruption of autophagy. Exemplary embodiments illustrating aspects of the present disclosure are described below in the context of several specific examples.

The term “subject” is used to refer to an animal (including a human and a non-human animal) to which the present apparatus and method may be applied. The term “user” is used to refer to a human that applies the device to a human or a non-human animal. The subject and the user may be the same human person, but not necessarily so.

Referring now to FIG. 1, an illustrative embodiment of V-ATPase is shown. V-ATPase, also called vacuolar-type ATPase, is a type of ATPase that utilizes the energy from ATP hydrolysis to transport protons across cellular membranes. It should be noted that V-ATPase is made up of several subunits. Mutations in one or more of these subunits, or mutations in non-coding regions causing a subunit not to be produced efficiently, may cause V-ATPase to fail to assemble properly at normal levels, and/or may cause V-ATPase not to function properly. V-ATPase subunits include ATP6AP1, ATP6V0E1, ATP6V0E2, ATP6V0D1, ATP6V0D2, ATP6V0B, ATP6V0C, ATP6V0A1, ATP6V0A2, ATP6V0A4, ATP6VIH, ATP6V1A, ATP6VIB1, ATP6V1B2, ATP6V1C1, ATP6VIC2, ATP6VID, ATP6V1E1, ATP6V1E2, ATP6V1G1, ATP6V1G2, ATP6V1G3, and ATP6VIF. V-ATPase subunits include V₀ subunits 104 and V₁ subunits 108. V₀ may include a portion of V-ATPase 100 responsible for transferring protons from one side of a membrane to the other. V₁ 108 may include a portion of V-ATPase 100 responsible for hydrolyzing ATP is shown. In some embodiments, V₁ 108 may receive ATP from other organelles of a cell. In particular, mitochondria may produce ATP to send to various organelles and proteins of a cell. Further, ATP may be used as an energy source for V-ATPase 100 to function properly. A properly functioning V-ATPase 100 may facilitate H+ (a proton) transfer across a membrane. In some instances, transferring protons across a membrane 112 may be an active transport that may require an energy source (e.g., ATP) to facilitate the active transport. Active transport may be employed because of a higher concentration of protons in an extracellular environment adjacent to the cell membrane 112. In some instances, V-ATPase 100 may function as an assembly factor and/or an ion channel stabilization factor.

Still referring to FIG. 1, ATPase H+ transporting accessory protein 2 (ATP6AP2) is a protein associated with V-ATPase. In some embodiments, a mutation in a coding or non-coding region associated with ATP6AP2 may cause V-ATPase to fail to assemble and/or function properly.

Still referring to FIG. 1, V-ATPase is an enzyme that uses energy from ATP to push protons (i.e. H+ ions) across a membrane, upstream of a gradient, thereby generating a pH or acidity level, and/or electrostatic charge, that is necessary for organelle function. Improper cellular acidity can be lethal or debilitating to an organism on a large-scale level. On a smaller-scale level, each organelle within an animal cell may have a specific pH to perform its own function. Thus, improper or suboptimal pH in cells or organelles can be harmful to an organ, a cell, or other portions of an organism in various ways. V-ATPases may be located on the membranes of organelles to regulate their pH and generate an acidity that facilitates various functions but are commonly referred to as lysosomal accessory proteins as they express heavily on lysosomal membranes.

Still referring to FIG. 1, V-ATPase 100 may include a transmembrane protein. Transmembrane proteins span from an internal portion of a cell or organelle to an external portion of the cell or organelle. It should be noted that transmembrane proteins may transport any substance across a cell or organelle membrane against a concentration gradient, transport large substances through the cell or organelle membrane, or any process of the like. As a non-limiting example, a portion of a V-ATPase 100 may be disposed in a hydrophobic region of membrane 112. As illustrated in FIG. 1, V₀ 104 of V-ATPase 100 may be disposed in membrane 112. V₀ 104 may span from hydrophilic heads in an internal portion of a cell or organelle to hydrophilic heads in an external portion of the cell or organelle. In some embodiments, a V₀ 104 portion of V-ATPase 100 may include ATP6AP2 which is in an assembly factor in V-ATPase 100.

Still referring to FIG. 1, a properly functioning V-ATPase 100 may be described as a proton pump. A proton pump alters the acidity of cell or organelle environments by actively transporting protons across membranes. V-ATPase may be found on cell membranes and a variety of organelle membranes such as endoplasmic reticulum, Golgi apparatus, and lysosome membranes. A “lysosome” as used in this disclosure is any membrane-bound organelle responsible for breaking down macromolecules, cellular waste, and pathogens. Lysosomes may ensure proper cellular homeostasis, metabolism, and degradation of biomolecules.

Failure of V-ATPase to form at proper levels on these membranes may cause the organelles to have limited effectiveness in some functions, as described in more detail below.

Still referring to FIG. 1, in efforts to maintain optimal pH levels within a cell, cells may include a proton pump integrated into the cell's membrane to actively transports protons in and out of the cell. In some embodiments, a pH level within a cell may be too high (i.e., high [H+]), causing an imbalance for organelles within the cell. The proton pump may then be used to pump protons out of the cell to dilute the [H+] and lower the pH level to make it more optimal for cell functionality. Proton pumps may use energy in the form of adenosine triphosphate (ATP) to phosphorylate subunits and assemble the functional pump. This pump may alter acidity to create the proper pH environments within organelle space or cell exteriors which are needed to carry out a wide range of cellular processes.

Still referring to FIG. 1, an ATP6AP2 116 deficiency may cause altered pH environments within organelle spaces or cell exteriors, including lysosomes. The ATP6AP2 gene codes for a subunit of the V-ATPase protein complex. This protein is also known as the ER-localized transmembrane adaptor and lysosomal interacting protein due to the high levels of expression on Endoplasmic Reticulum and Lysosomal membrane. Genetic mutations of ATP6AP2 can cause multiple disorders including parkinsonism with spasticity, renal toxicity, epilepsy, neurodevelopmental issues, brain atrophy, encephalopathy, and X-linked or congenital disorder of glycosylation type IIr. In some embodiments, an individual may have multiple malfunctions, such as mutations, in the V-ATPase protein complex. In some embodiments, compositions and methods described herein may be used to treat one or more malfunctions in V-ATPase.

Still referring to FIG. 1, in some embodiments, a mutation in a V-ATPase subunit, such as ATP6AP2, may cause V-ATPase to fail to properly assemble in sufficient quantities on lysosomes. Lysosomes are important to the clearance of cellular waste products due to their function in autophagy. In autophagy, an autophagosome containing waste products fuses with a lysosome, which under normal circumstances contains enzymes and low pH. This combination, under normal circumstances, causes the waste products in the autophagosome to be degraded. However, if the lysosome membrane does not contain sufficient properly functioning V-ATPase complexes, the pH of the lysosome may be too high, such that when an autophagosome fuses with a lysosome, the waste products are not efficiently degraded.

Still referring to FIG. 1, organellular acidity is crucial for the waste management systems in animal cells. Lysosome and peroxisomes degrade waste through the use of digestive enzymes that activate in a specific pH environment. Loss of lysosome pH is seen in the category of diseases called Lysosomal Storage Disorders.

Still referring to FIG. 1, autophagy is a metabolic process that selectively or generally gathers and breaks down cellular waste products or improperly synthesized macromolecules. Autophagy may involve numerous signaling and scaffolding proteins that create the membrane structure that collects waste called the autophagosome. Lysosomes are membrane bound organelles that contain a pH and enzymes that degrade, break down, and/or denature waste. During autophagy, an autophagosome may fuse to a lysosome to allow for contents of the autophagosome to be degraded. In this way, autophagy is a regulative process in cells, disposing of mistakes and errors in cellular synthesis. As a metabolic process, it is necessary for the breakdown of waste to occur for new, proper synthesis to take place. In autophagic disorders, back-up of waste that cannot be degraded impacts regeneration and synthesis of new material. TFEB, the lysosomal synthesis pathway, promotes autophagy by binding to a promoter region of several autophagy genes.

Still referring to FIG. 1, enzymes in the lysosome may include cathepsins, collagenases, peptidases, nucleases, phosphatases, glycoprotein/lipid/oligosaccaride-sidases and lipases. This organelle can shape degradation by regulating signaling pathways such as mTOR, known as the regulating pathway of autophagy. Lysosomes may interact with the AMPK pathway that inhibits mTORC1. Lysosomes may regulate their own production through the TFEB pathway, a transcription factor pathway that induces the biogenesis of lysosomes. Lysosome function may be regulated by a V-ATPase proton pump. Lysosomes may serve many functions, including waste degradation and regulation of autophagy. A lysosome may require a particular pH and/or pH within a particular range to function optimally; as a non-limiting example, a range may include a pH anywhere between approximately 4.5 and 5. When pH of a lysosome is outside this ideal range, one or more functions that a lysosome performs may become altered. An optimal pH of a lysosome may be different from that of other components of a cell, such as the cytosol where pH may typically be around 7.2. This difference in pH may create a membrane electrical potential which may aid in proton transport across a cell, between organelles, or the like. When the pH of the cytosol and/or lysosome becomes altered, this can lead to a cascade of issues including cellular components building up inside the cell, inability to deliver building blocks of macromolecules of cells, loss of neuron function and many other issues. Lysosome functioning may also aid in regulating autophagy as part of a feedback pathway by providing building blocks of macromolecules. In some instances, this may eventually cause the transcription factor EB (TFEB) pathway to turn off lysosome production and eventually lead to disorders including but not limited to cancer, cardiovascular disease, neurodegeneration, infections, and/or aging.

Still referring to FIG. 1, V-ATPase deficiency may cause altered pH levels that may disrupt protein synthesis and/or glycosylation. Proteins may be synthesized using RNA in the Endoplasmic Reticulum. Synthesized proteins may be modified for stability, transport, and function by the Golgi Apparatus. Under normal pH conditions in the Golgi apparatus, sialyltransferases may attach sialic acids to proteins in a process known as glycosylation. Proper glycosylation may benecessary for proper protein function, such as by allowing them to adhere to certain targets in the body. In some instances, glycosylation may be needed for proteins to fold correctly. However, where pH is abnormal due to dysfunctional V-ATPase, proteins may not be glycosylated efficiently. In some embodiments, above normal pH in the Golgi apparatus may affect the distribution of sialyltransferases in the Golgi apparatus, which may impact glycosylation rates. Improper glycosylation is known as a category of diseases called Congenital Disorders of Glycosylation (CDG).

Still referring to FIG. 1, congenital disorders of glycosylation, such as those due to V-ATPase deficiency, may affect a subject's ability to properly form glycolipids as well. Glycolipids act as cellular recognition units whereas glycoproteins act as receptors for chemical signals. Reduction or loss of these macromolecules may result in depletion of proper cellular communication. There are diverse groups of glycoproteins that occur or express in varying levels throughout different organs in the body. The kind of glycosylation disorder that a person inherits may impact the disease phenotypes seen.

Now referring to FIG. 2, a block diagram of an exemplary composition 200 for treating a V-ATPase malfunction is illustrated. Composition 200 may be employed to remedy a malfunction within V-ATPase 100. Composition 200 may include a glycosylation precursor 204, a modulator 208, and a delivery vehicle 216. As described further below, in some embodiments, composition 200 may be used to treat a subject in need thereof, such as a subject suffering from a genetic disorder that causes insufficient production of functional V-ATPase.

Still referring to FIG. 2, composition 200 may include a glycosylation precursor. In some embodiments, a glycosylation precursor may modulate glycosylation. In some embodiments, a glycosylation precursor may promote glycosylation. In some embodiments, a glycosylation precursor may modulate lysosome pH. In some embodiments, a glycosylation precursor may raise lysosome pH. In some embodiments, a glycosylation precursor may lower lysosome pH. In some embodiments, a glycosylation precursor may include a sialic acid precursor. A sialic acid precursor may include any sialic acid precursor as described herein, including but not limited to ManNR, ManNAc, UDP-GlcNac, UDP-GlcNR, ManNac-6-P, and/or SiaNR. In some embodiments, glycosylation precursor 204 may make up at least one percent of composition 200.

Still referring to FIG. 2, composition 200 may include a modulator. As used herein, a “modulator” is matter that modulates glycosylation, modulates lysosome pH, or both. In some embodiments, a modulator may include a lysosome pH modulator. In some embodiments, a lysosome pH modulator may raise lysosome pH. In some embodiments, a lysosome pH modulator may lower lysosome pH. In some embodiments, a modulator may include a glycosylation modulator. In some embodiments, a glycosylation modulator may increase glycosylation rates. In some embodiments, a modulator may include a P2Y12 inhibitor. As used herein, a “P2Y12 inhibitor” is a substance that reduces ADP binding to P2Y12. In some embodiments, a P2Y12 inhibitor may modulate pH in lysosomes. In some embodiments, a P2Y12 inhibitor may reduce pH in lysosomes. In some embodiments, a P2Y12 inhibitor may modulate pH in lysosomes by increasing cAMP levels. In some embodiments, a drug other than a P2Y12 inhibitor that increases CAMP levels may be used as a modulator. In some embodiments, modulating lysosome pH in a subject that lacks sufficient functional V-ATPase complexes may allow lysosomes to more effectively cause waste products to be degraded in autophagy. In some embodiments, a lysosomal pH modulator may also modulate pH in the Golgi apparatus and endoplasmic reticulum. In some embodiments, a lysosomal pH modulator may be used to modulate organelle pH in the liver, kidney, heart, brain, and/or spinal cord. In some embodiments, a combination of glycosylation precursor 204 and modulator 208 may have a synergistic effect in increasing glycosylation rates. In some embodiments, a combination of glycosylation precursor 204 and modulator 208 may increase glycosylation rates more than either glycosylation precursor 204 or modulator 208 alone. In some embodiments, a combination of glycosylation precursor 204 and modulator 208 may have a synergistic effect in reducing lysosome pH. In some embodiments, a combination of glycosylation precursor 204 and modulator 208 may reduce lysosome pH more than either glycosylation precursor 204 or modulator 208 alone. Glycosylation precursor 204 and/or modulator 208 may individually and/or in combination synergistically augment each other's efficacy in reducing and/or treating any pathology described herein.

Still referring to FIG. 2, P2Y12 inhibitors inhibit binding of ADP to a P2Y12 receptor. P2Y12 inhibitor may attenuate platelet aggregation. In some instances, P2Y12 inhibitor may include any P2Y12 inhibitor as described herein, including but not limited to Clopidogrel (Plavix®), Prasugrel (Effient®), Ticlopidine (Ticlid®), and/or Ticagrelor (Brilinta®). In some embodiments, a lysosomal pH modulator may include a suitable blood thinner. P2Y12 inhibitor may make up at least one percent of composition 200. P2Y12 inhibitor may be used to improve pH in retinal cell lysosomes, improve pH environments surrounding GA, ER, and lysosomal pH in multiple tissues. A P2Y12 inhibitor may affect restoring lysosomal pH in retinal cells in age-related macular degeneration. Macular degeneration may include similar characteristics as many autophagy-related diseases including accumulation of deposits in the Bruch's membrane of the eye. These deposits may include a toxic “clogging” effect that leads to the degeneration of the retinal pigment endothelium (RPE). In a non-limiting example, composition 100 may manipulate the lysosomes of other cell types, may correct our defective pathway, and may improve the lysosomal storage phenotypes seen in an ATP6AP2 mutation.

Still referring to FIG. 2, glycosylation precursor 204 and modulator 208 such as P2Y12 inhibitor may be combined to yield a cocktail 212. Various Concentrations and amounts of glycosylation precursor 204 and modulator 208 such as P2Y12 inhibitor may be chemically combined in lab glassware. As used in this disclosure, “novel cocktail” is a chemical combination of at least glycosylation precursor and P2Y12 inhibitor that is used as a treatment for an identified malfunction of a V-ATPase protein. Novel cocktail 212 may be used to deliver composition 200 to V-ATPase 100. In some instances, novel cocktail 212 may be used to treat ATP6AP2 116 deficiency. However, in some embodiments, novel cocktail 212 may be used to treat other deficiencies within a cell and/or organelle that may be negatively affecting a pH of the cell and/or organelle. In some embodiments, modulator 208 individually may be used to treat a V-ATPase malfunction. In some embodiments, glycosylation precursor 204 individually may be used to treat a V-ATPase malfunction. In some embodiments, modulator 208 and glycosylation precursor 204 may be used in combination to treat a V-ATPase malfunction. In some embodiments, a combination of modulator 208 and glycosylation precursor 204 may have a synergistic effect such as increasing glycosylation rates more than one element of the combination by itself and/or reducing lysosome pH more than one element of the combination by itself. Modulator 208 and glycosylation precursor 204 may be tested along and/or in combination.

Still referring to FIG. 2, composition 200 may include a delivery vehicle 216. As used herein, a “delivery vehicle” is an agent suitable for delivering a payload to a target site in a subject, including without limitation an organ, cell, and/or organelle. In some embodiments, a delivery vehicle may include a prodrug delivery system. In some embodiments, prodrug delivery system may include a prodrug. As used in this disclosure, a “prodrug” is a biologically inactive compound which can be converted, metabolized, or excreted leaving the active medication or therapeutic in the body within the organs/areas of interest. Prodrug strategy may enhance pharmacokinetic properties in a payload. The method of use application for this prodrug carrier may increase efficacy of the administered dosage. Prodrugs may be selected in order to target specific tissues or organs, potentially reducing off-target effects. In some embodiments, a prodrug delivery system may be capable of delivering a payload across the blood brain barrier. In some embodiments, a prodrug delivery system may allow glycosylation precursor 204 and/or modulator 208 to cross the blood brain barrier, without crossing itself. Prodrugs may include detachable units or bioreversible versions of the active drug that can be transformed in vivo by a chemical reaction that should occur in a predicted system to release the active compound. Prodrug may be used as a system of delivery in “hard to reach” organs including the central nervous system (CNS) and skeletal muscle. In CNS delivery constructs, the prodrug may include a lipophilic carbon chain that can be bound to endogenous proteins, carried in the blood, and eventually cross the blood-brain-barrier (BBB). In some embodiments, a first prodrug delivery system is used to deliver a lysosomal pH modulator and a second prodrug delivery system is used to deliver a glycosylation precursor. In some embodiments, a first prodrug delivery system is used to deliver both a lysosomal pH modulator and a glycosylation precursor.

Still referring to FIG. 2, prodrug may include a ProTide prodrug. ProTide prodrug is a category of prodrug that connects to a phosphate. Prodrug may be used with ManNAc-6-phosphate which is another glycosylation precursor. As a non-limiting example, a masked lipophilic ManNAc-6-P may go through enzymatic activation and enzymatic deacetylation to yield a ManNAc-6-P which is a precursor to sialic acid. Glycosylation precursors may be used in treatment of ATP6AP2 as discussed further in this disclosure.

Still referring to FIG. 2, prodrug delivery system may be a more efficient transport mechanism once sialic acid is added. As used in this disclosure, a “transport mechanism” is a process of moving substances into a cell or out of the cell. Prodrug delivery system may be more efficient because prodrug delivery system is able to deactivate a drug during biodistribution such that the drug is specifically delivered to its target. As described herein, ManNAc may be a glycosylation precursor 204. Therefore, having prodrug include ManNAc may provide a precursor to sialic acid such that when prodrug is metabolized, it may yield sialic acid and thus have a more efficient transport mechanism. Further, having prodrug including ManNAc may enable specific delivery of prodrug (including ManNAc) to its target. It should be noted that structure of prodrug may introduce delivery constraints. As a non-limiting example, prodrug having 16 carbons may cross a blood-brain-barrier (BBB). As another non-limiting example, prodrug having only 12 carbons may not cross a BBB.

Still referring to FIG. 2, administration of a glycosylation precursor 204 using a prodrug may increase glycosylation. In some embodiments, a V-ATPase 100 malfunction may cause a cell and/or organelle to have a pH environment that is not acidic enough, or too acidic, for sialic acid transfer. By delivering glycosylation precursor 204 using a prodrug, glycosylation precursor may still be delivered to a cell via the prodrug and facilitate the final steps of glycosylation in proteins.

Still referring to FIG. 2, prodrug may include any prodrug delivery system as described herein. In some embodiments, g-dopamine and/or g-adenosine receptors may be a part of the novel cocktail. G-dopamine receptors may be G protein-coupled receptors involved in regulating motor activity and various neurological disorders including but not limited to schizophrenia, bipolar disorder, Alzheimer's, Parkinson's, and the like. G-adenosine receptors may be G protein-coupled receptors involved in mediating the physiological actions of adenosine.

Still referring to FIG. 2, G-adenosine and/or G-dopamine receptors may alternatively or additionally be utilized to the phosphorylate assembly factors of V-ATPase 100. In some instances, it may not be desirable to employ a P2Y12 receptor to initiate pathways to phosphorylate assembly factors of V-ATPase 100. In an embodiment, other G-protein receptors (e.g., G-adenosine, G-dopamine) may be used to phosphorylate ATP6AP2 116 in order to remedy ATP6AP2 116 deficiency. In another embodiment, G-adenosine and/or G-dopamine may be used to remedy a deficiency in any other assembly factor of V-ATPase 100. Upon reading this disclosure, one of ordinary skill in the art would understand the assembly factors that may have deficiencies that can be remedied by G-protein receptors induced phosphorylation.

Still referring to FIG. 2, in some embodiments, a delivery vehicle may include an organelle targeting component. An organelle targeting component may direct the payload to an organelle of interest, such as a lysosome or Golgi apparatus. In a non-limiting example, a compound included in the makeup of an organelle of interest may be attached to a payload. In another non-limiting example, techniques common to enzyme replacement therapy such as interaction with mannose-6-phosphate may be used to target lysosomes.

Still referring to FIG. 2, composition 200 may be formulated in any dosage form, including but not limited to oral, ophthalmic, inhalation, injection, topical, intravaginal, and/or rectal administration. For instance and without limitation, composition 200 may be formulated as a pill, capsule, syrup, solution, elixir, emulsion, tincture, orally disintegrating tablet, lozenge, think film, powder, edible, eye drop, lotion, ointment, aerosolized medication, metered dose, nebulizer, smoking, vaporizer, intradermal, subcutaneously, intramuscularly, intraosseous, intraperitoneally, intravenously, cream, gel, hydrogel, ear drop, dermal patch, powder, and the like. In some embodiments, composition 200 may be administered using a dose schedule. As used in this disclosure, a “dose schedule,” is the amount of a composition to be used for a subject at a given time. In some embodiments, dosage schedule may include frequency, timing, duration, dosage, or the like. As a non-limiting example, dose schedule may include the time when the administration of composition 200 are to be given, the time between the administrations of composition 200, the length of time composition 200 are to be given, and/or the amount of composition 200 to be given at each specific time, respectively. In a non-limiting example, composition 200 may be administered daily, twice daily, 3 times daily, once every 6 months, or the like. In another non-limiting example, doses of Glycosylation precursor 204 may be administered daily and doses of P2Y12 inhibitor may be administered twice daily. In another non-limiting example, doses between 0.2 g to 10 g of Glycosylation precursor 204 may be dosed orally, intravenously, or by injection. In a non-limiting example, glycosylation precursor 204 may increase 60% transferrin to 85% transferrin (+25%) glycosylation. Additionally and/or alternatively, glycosylation precursor 204 may increase glycosylation from 5% to 75%. In another non-limiting example, P2Y12 inhibitor may increase pH in lysosomes from ˜7 to ˜4.8 (−2.2). Additionally and/or alternatively, P2Y12 inhibitor may decrease pH in lysosomes between 0.3 to 4.

Still referring to FIG. 2, composition 200 may be employed to address syndromic protein synthesis and to improve glycosylation effects on the liver. In addition, composition 200 may be utilized for glycosylation effects in protein synthesis, protein translation and glycoprotein interaction with various organelles in liver, kidney, heart, brain, and spinal cord.

Still referring to FIG. 2, composition 200 may be utilized to address deficient protein trafficking, degradation, and waste removal may be employed. Composition 200 may improve pH in retinal cell lysosomes. In some embodiments, composition 200 may downregulate enzymes, acidification, or both. For the purposes of this disclosure, “downregulation” is a decrease in the expression, production, or activity of a specific gene, protein, or receptor in a biological system. In some embodiments, composition 200 may upregulate enzymes, acidification, or both. For the purposes of this disclosure, an “upregulation” is an increase in the expression, production, or activity of a specific gene, protein, or receptor in a biological system. In a non-limiting example, composition may upregulate acidification to not only treat ATP6AP1 mutations, but mutations in lysosomal enzyme and glycosylation enzyme genes. Composition 200 may be utilized to improve GA, ER, and lysosomal pH in multiple tissues the tissues previously listed. Composition 200 may be utilized for congenital disorders of glycosylation (CDGs) and lysosomal storage disorders (LSCs). “Congenital disorders of glycosylation,” for the purposes of this disclosure, are a set of rare, genetic disorders that impact a person's ability to build some or all of their glycoproteins and glycolipids. Glycolipids can act as cellular recognition units whereas glycoproteins can act as receptors for chemical signals. Reduction or loss of these macromolecules can result in depletion of proper cellular communication. There are diverse groups of glycoproteins that can occur or express in varying levels throughout different organs in the body. The kind of glycosylation disorder that a person inherits can impact the disease phenotypes seen. Largely, most CDG patients experience: hypotonia, failure to thrive or grow, developmental delays, liver disease, bleeding or clotting disorders, seizures, strokes, cardiomyopathy, ataxia, hormonal imbalances, muscular disease, muscular dystrophy, Duchenne muscular dystrophy, neuromuscular scoliosis, poor vision, muscular degeneration, dysarthria, and renal failure. Therapies that target improving glycosylation may have positive therapeutic effects in these patients. In a non-limiting example, this therapeutic may serve as a remedy for glycosylation by adding precursor molecules, improving proteinopathies by correcting protein synthesis and translation at the beginning of pathway. In a non-limiting example, composition 200 may affect ATP6AP2 asp 107asp C-T, ATP6AP2 ser115ser C-T, ATP6AP2IVS2DS T-A+6, ATP6AP2 leu98ser, ATP6AP2 arg71his, ATP6AP2 IVS3AS TT del, ATP6AP2 ile21met, or the like to treat ATP6AP2 deficiencies.

Still referring to FIG. 2, in some embodiments, this therapeutic may serve as a remedy for lysosomal acidification. Lysosomal degradation is the final step in clearing toxic proteins caused by improper synthesis or misfolding. Improving lysosomal acidification would correct the proteinopathy at the final checkpoint of the pathway before cell death occurs. A lipid-based prodrug delivery may be used to deliver composition 200. The lipid-based delivery system may be a construct consisting of a multi-carbon chain which may infiltrate muscle, tissue, and even brain. Defects in lysosomes can lead to a category of diseases called lysosomal storage disorders. These disorders are characterized by a loss of enzyme or loss of functionality of those enzymes. In acidification disorders, enzyme function in lysosomes can be impaired. This impairment can lead to toxic buildup in cells. These diseases may include, but are not limited to, Fabry disease, Niemman-Pick disease, Krabbe disease, Gaucher disease, Metachromatic leukodystrophy, Sandhoff disease, Tay-sachs disease, Batten disease, Cystinosis, Danon disease, Pompe disease, and others. Symptoms of these diseases may include abnormally large organs, changes in skeletal muscle, coarse facial features, and development problems. In some embodiments, composition 200 may be configured to treat lysosomal storage disorder; for instance, but not limited to Mucopolysaccharidoses (MPS) I, II, IIA, IIIB, IIIC, IIID, IVA, IVB, VI, VII, type IV Mucolipidosis (ML4) neurodegenerative lysosomal storage disease caused by mutations in TRPML1, multiple sulphatase deficiency, Fabry, Farber lipogranulomatosis, Gaucher, Krabbe, Niemann-pick A, B and C, GM1 gangliosidosis, GM2 gangliosidosis, Aspartylglucosaminuria, Fucosidosis, α-Mannosidosis, B-Mannosidosis, Sialidosis, Schindler, Pompe disease, or the like as described above. In a non-limiting example, composition 200 may affect GBA 1q21, GLA Xq22, GAA 17q25.3, IDUA 4p16.3, IDS Xq28, SGSH 17q25.3, NAGLU 17q21.2, HGSNAT 8p11.21, HGSNAT 8p11.21, GNS 12q14.3, GALNS 16p24.3, GLB1 3p22.3, ARSB 5q14.1, GUSB 7q11.21, SMPD1 11p15.4, NPC1, NPC 2, ASAHI 10q22.1, HEXA 15q23, HEXB 5q13, GALC 14q31.3, ARSA 22q13.33, SUFM1 3p26.1, AGA 4q34.3, FUCA1 1p36.11, MANSA 19p13.2, NAGA 22q13.2, NEU1 6p21.33, GNPTAB 12q23.2, TRMPL1 19p13.2, LIPA 10q23.31, CTSA 20q13.12, LAMP2A Xq24 6′-(R)-methyl-5-O-(5-amino-5,6-dideoxy-alpha-L-talofuranosyl)-paromamine sulfate, Gemfibrozil, N-t-butylhydroxylamine, Modified cholera toxin, Pyrimethamine, Ambroxol, N-acetyl-glucosamine thiazoline, Migalastat hydrochloride*, miglustat, Odiparcil, Lucerastat, Venglustat, (3S)-1-azabicyclo [2.2.2]oct-3-yl {2-[2-(4-fluorophenyl)-1,3-thiazol-4-yl] propan-2-yl} carbamate, 2-hydroxypropyl-B-cyclodextrin, Hydroxy-Propyl-Beta-Cyclodextrin, Eliglustat, Cysteamine, 1,5-(Butylimino)-1,5 dideoxy,D-glucitol, L-cycloserine, Clenbuterol, Ursodeoxycholic acid, Gemfibrozil and vitamin A, Ibudilast, Pentosan polysulfate sodium, Triheptanoin, or the like.

Still referring to FIG. 2, composition 200 may be utilized for other proton pump defects such as but not limited to liver inflammation and necrosis, neurodevelopment, muscular development, immune system responses, blood sugar and pressure, nerve maintenance, heart function, kidney fibrosis and renal malfunctions. Composition 200 may be utilized for faulty proton pump function that affects autophagy and lysosomes and manifests as degenerative diseases including but not limited to Alzheimer's, multiple sclerosis, spinal muscular atrophy, Parkinson's, Huntington's., cancer, Parkinsonism with spasticity, congenital disorder of glycosylation type IIr, Hedera type of X-linked syndromic intellectual developmental disorder, or the like. Composition 200 may be utilized for faulty proton pump function that affects glycosylation and manifests as congenital disorders of glycosylation including but not limited to 170 CDG subtypes, Type 2 Diabetes Mellites, metabolism, and/or obesity. Composition 200 may be utilized for faulty proton pump function that affects immune and inflammatory response and manifests as infectious disease including but not limited to bacterial defense, COVID-19, COPD, and/or cancer.

Still referring to FIG. 2, in some embodiments, composition 200 may be configured to treat CDGs; for instance, but not limited to congenital disorder of deglycosylation 1 & 2, congenital disorder of glycosylation Ia-z, IIa-z, or the like. In a non-limiting example, composition 200 may affect ALG6 ALA333VAL, ALG6 SER478PRO, ALG6 IVS3DS G-A, ALG6 3bp del 895ATA, ALG6 3bp del 897AAT, ALG6 IVS7DS T-G, ALG6 TYR131HIS, NGLY1 ARG401TER, NGLY1 1-bp dup 1370G, NGLY1 3bp del 1205TTC, NGLY1 ARG542TER, NGLY1 CYS283TRP, NGLY1 GLU356GLY, NGLY1, 1bp del NT1837, PMM2 ARG141HIS, PMM2 ASN216ILE, PMM2 VAL129MET, PMM2 ARG162TRP, PMM2 ASP65TYR, PMM2, PHE119LEU, PMM2, ASP188GLY, PMM2 GLY117ARG, PMM2 ASP223GLU, PMM2 357C-A, PMM2 THR237ARG,

PMM2 CYS241SER, PMM2 ILE132THR, PMM2 VAL231MET, PMM2 CYS9TYR, PMM2 LEU32ARG, PMM2 THR226SER, PMM2 PRO113LEU, PMM2 IVS7 C-T, PMM2 VAL44ALA, PMM2, 28-kb del, PMM2 IVS3ASAS G-C, PMM2 TYR106PHE,MGAT2 SER290PHE, MGAT2 HIS262ARG, MGAT2 ASN318ASP, MGAT2 CYS339TER, MGAT2 LYS237ASN, ALG12 PHE142VAL, ALG12 THR61MET, ALG12 ARG146GLN, ALG12 GLY101ARG, ALG12 LEU158PRO, ALG12 TYR414TER, ALG12 THR224MET, ALG12 1bp del 1001A, ALG12 1bp del 117G, SLC35C1 ARG147CYS, SLC35C1 THR308ARG, SLC35C1 GLU31TER, SLC35C1 3bp del 501CTT, ALGI SER258LEU, ALG1 GLU342PRO, ALGI SER150ARG, ALG1 MET377VAL, ALGI GLY145ASP, ALGI CYS396TER, ALGI ARG276TRP, MPI ARG219GLN, MPI SER102LEU, MPI MET138THR, MPI 1bp ins 166C, MPI ARG295HIS, DPM1 ARG92GLY, DPM1 13bp del, DPM1 1bp del 628C, DPM1 SER248PRO, DPM1 IVS4AS T-A, DPM1 GLY152VAL, DPM1 100kb del, STT3A VAL626ALA, STT3A THR546ILE, STT3A TYR530SER, STT3A HIS46ARG, STT3A ARG160GLN, STT3A ARG405CYS, STT3A ARG405HIS, STT3A ARG329CYS, or the like.

Still referring to FIG. 2, in some embodiments, composition 200 may be configured to treat other V-ATPase deficiencies; for instance, but not limited to Cutis laxa type 2, Developmental and Epileptic Encephalopathy 93, immunodeficiency 47, X-linked myopathy with excessive autophagy, Distal renal tubular acidosis 3, Developmental and Epileptic Encephalopathy 104, neurodevelopmental disorder with epilepsy and brain atrophy, Wrinkly skin syndrome, Distal renal tubular acidosis 2 with progressive sensorineural hearing loss, Congenital Deafness with onychodystrophy, Zimmermann-Laband syndrome 2, or the like. In a non-limiting example, composition 200 may affect ATP6AP1 met428ile, ATP6AP1 leu144pro, ATP6AP1 glu346lys, ATP6AP1 tyr313cys, ATP6AP1 leu181arg, ATP6AP1 leu47pro, ATP6AP1 leu311gln, ATP6AP1 tyr217asn, ATP6V1A ARG338CYS, ATP6V1A GLY72ASP, ATP6V1A ASP100TYR, ATP6V1A ASP349ASN, ATP6V1A PRO27ARG, ATP6V1A ASP371GLY, VMA21 IVS1 A-C, VMA21 IVS1 A-T, VMA21 IVS2 A-G, VMA21 IVS2 T-G, VMA21 272G-C, VMA21 TER+6, VMA21 IVS2 T-G, VMA21 92-bp del, VMA21 9-bp del, ATP6V1E1 LEU128PRO, ATP6V1E1 ARG212TRP, ATP6V0A4 GLU753TER, ATP6V0A4 GLY820ARG, ATP6V0A4 IVS17 G-A, ATP6V0A4 1 bp deVAL351, ATP6V0A4 MET580THR, ATP6V0A4 IVS6 G-A, ATP6V0A4 1bp de GLN2761, ATP6V0A4 PRO524LEU, ATP6V0A4 TYR502TER, ATP6V0A4 ARG807GLN, ATP6V0A4, 1bp del 2137G, ATP6V0A1 ARG741GLN, ATP6V0A1 ALA512PRO, ATP6V0A1 50-kb del, ATP6V0A1 ASN534ASP, ATP6V0A1, IVS2 G-A, ATP6V0A1 ARG740GLN, ATP6V0A1 GLY551GLU, ATP6V0A1 ARG804HIS, ATP6V0A1 1bp del 445G, AP6V0A1 ARG495TRP, ATP6V0A2 1bp ins 100A, ATP6V0A2 7bp del NT2355, ATP6V0A2 10132G-A, ATP6V0A2 ARG63TER, ATP6V0A2 GLN765TER, ATP6V1B1 IVS12DS G-C, ATP6V1B1GLY78ARG, ATP6V1B1 LEU81PRO, ATP6V1B1 IVS6DS G-A, ATP6V1B1 1bp del, ATP6V1B1ARG31TER, ATP6V1B2 ARG485PRO, ATP6V1B2 ARG506TER, or the like.

Still referring to FIG. 2, abnormal upregulation or downregulation can be a characteristic of multiple pathologies. In a non-limiting example, autophagy-related diseases may include neurodegenerative, aging, and metabolic diseases. Metabolic disorders linked to autophagy may include diabetes, obesity, non-alcoholic steatohepatitis, and atherosclerosis. Things like over-nutrition, high fat diets, insulin resistance, and high cholesterol can create or result in dysfunction within autophagy. Impaired autophagy can be found in Parkinson's disease, Alzheimer's disease, Huntington's disease, amyotrophic lateral sclerosis, and aging. Upregulation of autophagy on toxic protein can aggregate as a therapeutic solution to these neurodegenerative diseases. Such upregulators of autophagy may include starvation conditions, ALFY overexpression, Afinitor, Torisel, Zortress, Rapamune, Hyftor, Fyarro, Afinitor Disperz, Ridaforolimus, Umirolimus, Zotarolimus, Torin-1, Torin-2, Vistusertib, and others. In some embodiments, composition 200 may be configured to treat autophagic disorders; for instance, but not limited to Paget Disease of Bone 3, Frontotemporal Dementia, Amyotrophic Lateral Sclerosis 3, Distal Myopathy with Rimmed Vacuoles 2, childhood-onset neurodegeneration with ataxia, dystonia, gaze palsy, Alzheimer's, Parkinson's, or the like. In a non-limiting example, composition 200 may affect SQSTM1PRO392LEU, SQSTM1 1bp ins 1224T, SQSTM1 IVS7DS G-A, SQSTM1 LYS378TER, SQSTM1 PRO387LEU, SQSTM1 ALA33VAL, SQSTM1 3 bp del 714GAA, SQSTM1 2T-A, SQSTM1 2bp del NT311, SQSTM1 ARG96TER, SQSTM1 IVS2DS T-A, SQSTM1 ARG312TER, SQSTM1 1bp ins 875T, or the like.

Still referring to FIG. 2, in some embodiments, composition 200 may be configured to treat aging and/or neurodegeneration; for instance, but not limited to Macular degeneration, Cerebral arteriopathy, Stargardt disease, Retinitis Pigmentosa, Cone-rod dystrophy 3, Fundus Flavimaculatus, or the like. In a non-limiting example, composition 200 may affect C2 GLY444ARG, C2 GLU318ASP, C2 IVS10 G-T, CFB LEU9HIS, CFB ARG32GLN, CFB PHE286LEU, CFB PHE286LEU, CFB LYS323GLU, HTRA1 512G-A, HTRA1 ARG370TER, HTRA1 ARG302TER, HTRA1 VAL297MET, HTRA1 ALA252THR, HTRA1 GLY295ARG, HTRA1 ALA321THR, HTRA1 1bp del 126G, HTRA1 ARG166LEU, HTRA1 ALA173PRO, HTRA1 SER284ARG, HTRA1 IVS4AS G-A, ABCA4 GLY863ALA, ABCA4 VAL931MET, ABCA4 ALA1028VAL, ABCA4 LEU2027PHE, ABCA4 VAL2050LEU, ABCA4 ASP2177ASN, ABCA4 GLY1961GLU, ABCA4 1-bp del 1847A, ABCA4 IVS30DS G-T, ABCA4 IVS40DS G-A, ABCA4 TRP855TER, ABCA4 GLU1036LYS, ABCA4 2bp ins 3211GT, ABCA4 LEU1970PHE, ABCA4 LEU1971ARG, ABCA4 ALA1038VAL, ABCA4 IVS13AS G-A, ABCA4 TYR340ASP, ABCA4 IVS5AS A-G, ABCA4 ARG212CYS, ABCA4 ARG18TRP, ABCA4 ARG572GLN, ABCA4 LEU541PRO&ALA1038VAL, ABCA4 2bp del 2617CT, ABCA4, LEU1201ARG, ABCA4 PRO1380LEU, ABCA4 1bp del 2888G, ABCA4 1bp del 1225A, ABCA4 ARG2030TER, ABCA4 IVS39AS T-C, ABCA4 ALA1762ASP, ABCA4 15bp del NT3539, ABCA4 LEU1940PRO, ABCA4 PRO1780ALA, ABCA4 ARG943GLN, ABCA4 TRP821ARG, ABCA4 GLU1122LYS, or the like.

Still referring to FIG. 2, in some embodiments, composition 200 may be configured to treat metabolic disorders; for instance, but not limited to Thrombophilia 11 due to HRG deficiency, Leukocyte adhesion deficiency, or the like. In a non-limiting example, composition 200 may affect HRG GLY85GLU, HRG CYS223ARG, HRG PRO73SER, ITGB2 ARG593CYS, ITGB2 LYS196THR, ITGB2 LEU149PRO, ITGB2 GLY169ARG, ITGB2 ATG-AAG, ITGB2 ARG586TRP&12bp ins, ITGB2 ASN351SER, ITGB2 PRO178LEU, ITGB2 ASP128ASN, ITGB2 IVSDS G-A, ITGB2 GLY284SER, ITGB2 SER138PRO, ITGB2 GLY273ARG, ITGB2 IVS4AS 169bp del, or the like.

Still referring to FIG. 2, in some embodiments, improper pH in lysosomes as a result of insufficient functional V-ATPase may cause B cells to fail to produce sufficient antigens and/or may cause reduced B cell counts. This may reduce the effectiveness of the immune system of a subject suffering from such a disorder. In some embodiments, composition 200 may be used to improve the immune system of such a subject. In some embodiments, composition 200 may be administered to a subject with an infection, such as a bacterial or viral infection, or at increased risk of infection, where the subject has a disorder involving insufficient functional V-ATPase.

Still referring to FIG. 2, in some embodiments, composition 200 may be used to treat a viral infection in a subject with insufficient functional V-ATPase. Such infections may include, but are not limited to influenza, Encephalomyocarditis virus, Hepatitis C virus, respiratory syncytial virus, human immunodeficiency virus-1, human rhinovirus, Zika virus, Dengue virus, Rift valley virus, Measles, Sendai virus, enterovirus 71, Coronavirus, an infection of Helicobacter pylori or Mycobacterium tuberculosis, an infection of Mycobacterium tuberculosis, or the like. In a non-limiting example, composition 200 may affect NSP6, NLRP3 activation, or the like.

Still referring to FIG. 2, in some embodiments, composition 200 may be used to treat a bacterial infection in a subject with insufficient functional V-ATPase. Such infections may include, but are not limited to bacterial activated thrombosis, immune thrombocytopenia, or the like. In a non-limiting example, composition 200 may affect platelet adhesion, platelet aggregation, or the like. In some embodiments, composition 200 may be configured to treat an atherosclerotic disease, inflammatory bowel disease, oncological disease, or the like. In some embodiments, composition 200 may be configured to modulate Transient Receptor Potential Mucolipin-1 (TRPML1), Two-pore channels (TPCs), Transient Receptor Potential Mucolipin-3 (TRPML3), P2X₄, or the like. “Transient Receptor Potential Mucolipin-1,” as used in this disclosure, is a Ca²⁺ channel in the lysosome that regulates certain aspects of lysosome trafficking, including autophagy. TRPML1 is an inwardly rectifying current channel that transports cations from the lumen of the lysosome to the cytosol.

Release of Ca 2+ from the lysosome using TRPML1 can modulate transcription factor end binding activity using local calcineurin activation, which may induce autophagy and lysosomal biogenesis. Composition 200 may aid in avoiding Ca 2+ leaking from lysosomes via modulation of calcium transport across cell membranes. Composition 200 activates lysosomal biogenesis through improvement of V₀ and V₁ assembly and recruitment at a proton pump. Subunit C of V-ATPase pump may aid in holding V₀ and V₁ subunits together. In some embodiments, SidK may aid in identifying Subunit C activity. Composition 200 may impact subunit C of V-ATPase pump, driving increased assembly and expression of V₀ and V₁ subunits. Supplementation with subunit C may aid in increasing V₀ and V₁ assembly. “Lysosomal biogenesis” as used in this disclosure, is any process of formation, maturation, and maintenance of lysosomes. Lysosomal biogenesis may include gene regulation via transcription factor EB (TFEB), a key regulator of the autophagy-lysosomal pathway, which helps with cell break down and recycling. Upon activation, TFEB enters a cell nucleus and promotes the expression of genes involved in lysosome formation, function, and autophagy. TFEB dysregulation may produce overt neurological effects such as higher tau pathology and diminished microglia response caused by insufficient lysosomal acidification. Regulation of TFEB may aid in reversing neurological symptoms seen with V-ATPase malfunction. Regulation of TFEB may aid in microglia function producing neuroprotective effects. Microglia functions to provide neuroprotective function by removing cellular waste, debris and toxic protein aggregates by phagocytosis and autophagy, which depend on proper lysosomal acidification. Improvement of lysosomal biogenesis via improvement of microglia cell clean up function may improve the pathogenesis of Alzheimer's disease and other clinical presentations demonstrating tau pathology such as Down Syndrome's and lysosomal storage disorders. Lysosomal biogenesis may include vesicle trafficking and endosome maturation. Lysosomes originate from the Golgi apparatus, where they fuse with endosomes to form lysosomes. Golgi-derived vesicles may traffic and carry enzymes such as proteases, lipases, and glycosidases, which are essential for lysosomal function. Lysosomal biogenesis may include enzyme delivery via Mannose-6-Photphate (M6P) tagging. Lysosomal hydrolases are synthesized in the endoplasmic reticulum (ER) and tagged with M6P in the Golgi. The M6P receptor directs these enzymes to lysosomes. Lysosomal biogenesis may include lysosomal acidification and maturation. Lysosomes require an acidic environment with a pH of approximately 4.5-5 for optimal enzyme activity. V-ATPase proton pump helps maintain this acidic environment by actively transporting protons into a lysosome. Lysosomal biogenesis may aid in V-ATPase proton pump functionality as well as regenerative downstream effects that may be influenced by V-ATPase malfunction. Lysosomal biogenesis may include fusion with autophagosomes and endocytosed material to aid in degradation and recycling of cellular components. Lysosomal biogenesis may influence apolipoprotein E (APOE) gene which plays a role in lipid metabolism and neurodegenerative diseases. Different APOE isoforms may influence lysosomal function leading to cellular stress, impaired waste clearance and neurodegeneration. For instance and without limitation, APOE4 may cause defective cholesterol transport, leading to lipid accumulation in lysosomes thus ultimately disrupting the lysosomal membrane and ultimately affecting enzyme function and cellular clearance. APOE4 may impair lysosomal biogenesis, leading to defective autophagic flux and accumulation of toxic aggregates such as Beta-amyloid and tau proteins. APOE4 may also cause TFEB dysregulation by downregulating TFEB and thus impairing lysosomal and autophagy genes. APOE4 may exacerbate oxidative stress, damaging lysosomal membranes and causing lysosomal leakage. Leakage may release cathepsins (proteolytic enzymes) thus triggering neuroinflammation and neuronal death. APOE4 may also activate microglia causing debris and toxic protein aggregation as described above in more detail. APOE4 may weaken lysosomal degradation of Beta-amyloid plaques, contributing to pathogenesis of Alzheimer's disease as described above. Restoration of lysosomal function such as through administration of composition 200 may aid in enhancing lysosomal function via TFEB activation, increasing acid hydrolase activity to restore lysosomal degradation, promoting autophagy and lysosomal biogenesis by mammalian target of rapamycin (mTOR) activation as described in more detail below, and reducing lysosomal stress and inflammation. These neuroprotective mechanisms aid to remove cellular waste, debris, and toxic protein aggregates by phagocytosis and autophagy which depend on optimal lysosomal acidification.

Still referring to FIG. 2, composition 200 may be utilized to address pH in metabolic pathways such as but not limited to myopathy, hypertension, diabetes, tubular acidosis, chronic kidney disease, fibrosis, and/or cirrhosis. In a non-limiting example, composition 200 may be utilized to address pH in CNS pathways by acidification using V-ATPase 100 through organelle, such as, without limitation, lysosome and Golgi apparatus using waste removal and protein synthesis mechanism through organ system, such as, without limitation, heart, kidney, liver, or the like to address diseases disclosed above. As a non-limiting example, affected metabolic pathways may include interleukin secretion, CD36 translocation, inflammasome activity, endocytic trafficking, HGFR interaction, mTORC1 activation, or the like.

Still referring to FIG. 2, composition 200 may be utilized to address pH in central nervous system (CNS) pathways including but not limited to retinopathy, degeneration, gangliosidosis, encephalopathy, development, ataxia, stenosis, and/or sclerosis. In a non-limiting example, composition 200 may be utilized to address pH in CNS pathways by acidification using V-ATPase 100 through organelle, such as, without limitation, lysosome and Golgi apparatus using waste removal and protein synthesis mechanism through organ system, such as, without limitation, eyes, brain, spinal cord, or the like to address diseases disclosed above. As a non-limiting example, affected CNS pathways may include β/β-crystallin expression, notch signaling, Ac45RP neurite expansion, catalyzing palmitoyl transferase, polarization of neurons, or the like.

Still referring to FIG. 2, composition 200 may be utilized to address pH in immunological pathways including but not limited to coronavirus, RSV, influenza, HIV, staphylococcus, cancer immunogenicity, thrombocytopenia, and/or encephalomyocarditis. In a non-limiting example, composition 200 may be utilized to address pH in CNS pathways by acidification using V-ATPase 100 through organelle, such as, without limitation, lysosome and Golgi apparatus using waste removal and protein synthesis mechanism through organ system, such as, without limitation, heart, kidney, liver, or the like to address diseases disclosed above. As a non-limiting example, affected immunological pathways may include platelet aggregation, inflammasome activity, mTOR interaction, or the like.

Still referring to FIG. 2, composition 200 may address additional pH imbalances for other pathways. As a non-limiting example, composition 200 may address additional pH imbalances for ubiquitin-proteasome pathway. For the purposes of this disclosure, an “ubiquitin-proteasome pathway,” is a vital cellular mechanism responsible for regulating the degradation and turnover of proteins within eukaryotic cells. The ubiquitin-proteasome pathway may maintain cellular homeostasis, remove damaged or misfolded proteins, and control the levels of various proteins involved in cell cycle regulation, signal transduction, and other cellular processes.

Still referring to FIG. 2, in some embodiments, ATP6AP2 116 deficiency may include but is not limited to Parkinsonism with spasticity, congenital disorders of glycosylation type Iir, Hedera type of-X linked syndromic intellectual developmental disorders and the like. Lysosomes may serve many functions, including waste degradation and regulation of autophagy. A lysosome may require a particular pH to function optimally such as anywhere between 4.5-5. When the pH of a lysosome is outside this ideal range, one or more functions that a lysosome performs may become altered. The optimal pH of a lysosome may be different from other components of a cell, such as the cytosol where the pH may typically be around 7.2. This difference in pH may create a membrane potential which aids in proton transport across the cell. When the pH of the cytosol and/or lysosome becomes altered, this can lead to a cascade of issues including cellular components building up inside the cell, inability to deliver building blocks of macromolecules of cells, loss of neuron function and many other issues. Lysosome functioning may also aid in regulating autophagy as part of a feedback pathway by providing building blocks of macromolecules. In some instances, this may eventually cause the transcription factor EB (TFEB) pathway to turn off lysosome production and eventually lead to disorders including but not limited to cancer, cardiovascular disease, neurodegeneration, infections, and/or aging.

Still referring to FIG. 2, in some embodiments, composition 200 may comprise. 0.001% modulator. In some embodiments, composition 200 may comprise about 0.01% modulator by weight. In some embodiments, composition 200 may comprise about.1% modulator by weight. In some embodiments, composition 200 may comprise about 1% modulator by weight. In some embodiments, composition 200 may comprise about 5% modulator by weight. In some embodiments, composition 200 may comprise about 10% modulator by weight. In some embodiments, composition 200 may comprise about 15% modulator by weight. In some embodiments, composition 200 may comprise about 20% modulator by weight. In some embodiments, composition 200 may comprise about 30% modulator by weight. In some embodiments, composition 200 may comprise about 40% modulator by weight. In some embodiments, composition 200 may comprise about 50% modulator by weight. In some embodiments, composition 200 may comprise about 60% modulator by weight. In some embodiments, composition 200 may comprise about 70% modulator by weight. In some embodiments, composition 200 may comprise about 80% modulator by weight. In some embodiments, composition 200 may comprise about 90% modulator by weight. In some embodiments, composition 200 may comprise about 99% modulator by weight. In some embodiments, the percent of modulator within composition 200 may be within a range between above described values.

Still referring to FIG. 2, in some embodiments, composition 200 may comprise. 001% modulator. In some embodiments, composition 200 may comprise about 0.01mol %. In some embodiments, composition 200 may comprise about. 1 mol % modulator. In some embodiments, composition 200 may comprise about 1 mol % modulator. In some embodiments, composition 200 may comprise about 5 mol % modulator. In some embodiments, composition 200 may comprise about 10 mol % modulator. In some embodiments, composition 200 may comprise about 15 mol % modulator. In some embodiments, composition 200 may comprise about 20 mol % modulator. In some embodiments, composition 200 may comprise about 30 mol % modulator. In some embodiments, composition 200 may comprise about 40 mol % modulator. In some embodiments, composition 200 may comprise about 50 mol % modulator. In some embodiments, composition 200 may comprise about 60 mol % modulator. In some embodiments, composition 200 may comprise about 70 mol % modulator. In some embodiments, composition 200 may comprise about 80 mol % modulator. In some embodiments, composition 200 may comprise about 90 mol % modulator. In some embodiments, composition 200 may comprise about 99 mol % modulator. In some embodiments, the percent of modulator within composition 200 may be within a range between above described values.

Still referring to FIG. 2, in some embodiments, composition 200 may comprise. 0.001% glycosylation precursor. In some embodiments, composition 200 may comprise about. 0.01% glycosylation precursor by weight. In some embodiments, composition 200 may comprise about 0.1% glycosylation precursor by weight. In some embodiments, composition 200 may comprise about 1% glycosylation precursor by weight. In some embodiments, composition 200 may comprise about 5% glycosylation precursor by weight. In some embodiments, composition 200 may comprise about 10% glycosylation precursor by weight. In some embodiments, composition 200 may comprise about 15% glycosylation precursor by weight. In some embodiments, composition 200 may comprise about 20% glycosylation precursor by weight. In some embodiments, composition 200 may comprise about 30% glycosylation precursor by weight. In some embodiments, composition 200 may comprise about 40% glycosylation precursor by weight. In some embodiments, composition 200 may comprise about 50% glycosylation precursor by weight. In some embodiments, composition 200 may comprise about 60% glycosylation precursor by weight. In some embodiments, composition 200 may comprise about 70% glycosylation precursor by weight. In some embodiments, composition 200 may comprise about 80% glycosylation precursor by weight. In some embodiments, composition 200 may comprise about 90% glycosylation precursor by weight. In some embodiments, composition 200 may comprise about 99% glycosylation precursor by weight. In some embodiments, the percent of glycosylation precursor within composition 200 may be within a range between above described values.

Still referring to FIG. 2, in some embodiments, composition 200 may comprise. 0.001% glycosylation precursor. In some embodiments, composition 200 may comprise about. 0.01mol %. In some embodiments, composition 200 may comprise about 0.1 mol % glycosylation precursor. In some embodiments, composition 200 may comprise about 1 mol % glycosylation precursor. In some embodiments, composition 200 may comprise about 5 mol % glycosylation precursor. In some embodiments, composition 200 may comprise about 10 mol % glycosylation precursor. In some embodiments, composition 200 may comprise about 15 mol % glycosylation precursor. In some embodiments, composition 200 May comprise about 20 mol % glycosylation precursor. In some embodiments, composition 200 may comprise about 30 mol % glycosylation precursor. In some embodiments, composition 200 may comprise about 40 mol % glycosylation precursor. In some embodiments, composition 200 may comprise about 50 mol % glycosylation precursor. In some embodiments, composition 200 may comprise about 60 mol % glycosylation precursor. In some embodiments, composition 200 may comprise about 70 mol % glycosylation precursor. In some embodiments, composition 200 may comprise about 80 mol % glycosylation precursor. In some embodiments, composition 200 may comprise about 90 mol % glycosylation precursor. In some embodiments, composition 200 may comprise about 99 mol % glycosylation precursor. In some embodiments, the percent of glycosylation precursor within composition 200 may be within a range between above described values.

Still referring to FIG. 2, in some embodiments, composition 200 may comprise. 0.001% delivery vehicle. In some embodiments, composition 200 may comprise about 0.01% delivery vehicle by weight. In some embodiments, composition 200 may comprise about 0.1% delivery vehicle by weight. In some embodiments, composition 200 may comprise about 1% delivery vehicle by weight. In some embodiments, composition 200 may comprise about 5% delivery vehicle by weight. In some embodiments, composition 200 may comprise about 10% delivery vehicle by weight. In some embodiments, composition 200 may comprise about 15% delivery vehicle by weight. In some embodiments, composition 200 may comprise about 20% delivery vehicle by weight. In some embodiments, composition 200 may comprise about 30% delivery vehicle by weight. In some embodiments, composition 200 may comprise about 40% delivery vehicle by weight. In some embodiments, composition 200 may comprise about 50% delivery vehicle by weight. In some embodiments, composition 200 may comprise about 60% delivery vehicle by weight. In some embodiments, composition 200 may comprise about 70% delivery vehicle by weight. In some embodiments, composition 200 may comprise about 80% delivery vehicle by weight. In some embodiments, composition 200 may comprise about 90% delivery vehicle by weight. In some embodiments, composition 200 may comprise about 99% delivery vehicle by weight. In some embodiments, the percent of delivery vehicle within composition 200 may be within a range between above described values.

Still referring to FIG. 2, in some embodiments, composition 200 may comprise. 0.001% delivery vehicle. In some embodiments, composition 200 may comprise about 0.01 mol %. In some embodiments, composition 200 may comprise about 0.1 mol % delivery vehicle. In some embodiments, composition 200 may comprise about 1 mol % delivery vehicle. In some embodiments, composition 200 may comprise about 5 mol % delivery vehicle. In some embodiments, composition 200 may comprise about 10 mol % delivery vehicle. In some embodiments, composition 200 may comprise about 15 mol % delivery vehicle. In some embodiments, composition 200 may comprise about 20 mol % delivery vehicle. In some embodiments, composition 200 may comprise about 30 mol % delivery vehicle. In some embodiments, composition 200 may comprise about 40 mol % delivery vehicle. In some embodiments, composition 200 may comprise about 50 mol % delivery vehicle. In some embodiments, composition 200 may comprise about 60 mol % delivery vehicle. In some embodiments, composition 200 may comprise about 70 mol % delivery vehicle. In some embodiments, composition 200 may comprise about 80 mol % delivery vehicle. In some embodiments, composition 200 may comprise about 90 mol % delivery vehicle. In some embodiments, composition 200 may comprise about 99 mol % delivery vehicle. In some embodiments, the percent of delivery vehicle within composition 200 may be within a range between above described values.

Still referring to FIG. 2, in some embodiments, a composition may be customized according to characteristics of a subject to which the composition is to be administered. For example, different V-ATPase malfunctions may cause different symptoms in a subject, necessitating different treatments. In some embodiments, the percent modulator, glycosylation precursor, and delivery vehicle in the composition may be customized according to the subject and/or the specific V-ATPase malfunction. For example, if the specific V-ATPase malfunction mainly impacts glycosylation, then the percent glycosylation precursor may increase. In another example, if the specific V-ATPase malfunction mainly impacts lysosome pH, then the percent modulator may increase.

Still referring to FIG. 2, in some embodiments, a pharmaceutical composition may be manufactured by receiving a glycosylation precursor, receiving a modulator, and combining the glycosylation precursor and the modulator with a delivery vehicle. In some embodiments, such a pharmaceutical composition may be suitable for treating a V-ATPase malfunction.

Referring now to FIG. 3, a method 300 for treating a V-ATPase malfunction is shown. At step 305, method 300 may include identifying a malfunction of at least a portion of a V-ATPase protein. Identification may be done by a computing device as described herein. It should be noted that a test sample may be used to identify a malfunction. In some instances, identification may occur in real-time by using invasive testing. Portion of V-ATPase protein may be any portion of V-ATPase protein as described herein. Step 305 may be implemented in accordance with FIGS. 1 and 2 without limitation.

Still referring to FIG. 3, method 300, at step 310, may include preparing a glycosylation precursor. It should be noted that preparation of glycosylation precursor may be any suitable lab preparation. One of ordinary skill in the art, upon reading this disclosure would understand methods of suitable lab preparation for glycosylation precursor. Glycosylation precursor may be any glycosylation precursor described herein. Step 310 may be implemented in accordance with FIGS. 1 and 2 without limitation. Three enzymatic steps may convert glucose into uridine diphosphate N-acetylglucosamine (UDP-GlcNAc), the biochemical precursor for the biosynthesis of Neu5Ac as well as other carbohydrates. The bifunctional enzyme UDP-GlcNAc 2-epimerase/ManNAc kinase (GNE) can convert UDP-GlcNAc into NAcetylmannosamine (ManNAc) via its epimerase domain (GNE) and subsequently produces ManNAc-6-phosphate using its kinase activity (MNK). ManNAc-6-phosphate can be converted into Neu5Ac-9-phosphate by the N-acetylneuraminate synthase (NANS) and dephosphorylated by Neu5Ac-9-P-phosphatase (NANP) to yield Neu5Ac. In the nucleus, Neu5Ac can be conjugated with cytidine monophosphate (CMP) by the CMP sialic acid synthase (CMAS) and transported into the Golgi system via the CMP-sialic acid transporter SLC35A1. Cytosolic CMP-sialic acid levels can regulate the synthesis of sialic acids by feedback inhibition of GNE. In the Golgi, 20 sialyltransferase isoenzymes link sialic acid to glycans.

Sialoglycans present at the cell surface or in intracellular compartments like lysosomes can be cleaved by sialidases to release free sialic acids. Free lysosomal sialic acids can be transported into the cytosol via the sialin (SLC17A5) transporter for recycling in the sialic acid biosynthesis pathway or degradation into ManNAc and pyruvate by N-acetylneuraminate pyruvate lyase (NPL).

Still referring to FIG. 3, method 300, at step 315, may include preparing a modulator. In some embodiments, a modulator may include a P2Y12 inhibitor. It should be noted that preparation of P2Y12 inhibitor may be any suitable lab preparation. One of ordinary skill in the art, upon reading this disclosure would understand methods of suitable lab preparation for P2Y12 inhibitor. P2Y12 inhibitor may be any P2Y12 inhibitor described herein. Step 315 may be implemented in accordance with FIGS. 1 and 2 without limitation.

Still referring to FIG. 3, method 300, at step 320, may include combining glycosylation precursor and the modulator to yield a novel cocktail. One of ordinary skill in the art, upon reading this disclosure would understand methods of suitable lab preparation and conditions to combine glycosylation precursor and P2Y12. Conditions may include but are not limited to pH environment, temperature, pressure, volume, and the like. P2Y12 inhibitor and glycosylation precursor may be any P2Y12 inhibitor and glycosylation precursor as described herein. Step 320 may be implemented in accordance with FIGS. 1 and 2 without limitation.

Still referring to FIG. 3, method 300, at step 325, may include delivering novel cocktail. Delivering novel cocktail may be done in a controlled lab environment using a test sample. In some embodiments, delivering novel cocktail may be done invasively. Novel cocktail may be any novel cocktail as described herein. In some instances, delivery of novel cocktail may be carried out by a computing device. Computing device may receive at least an input from a user indicating test parameters. Test parameters may include time, temperature, pH, and the like. Computing device may be any computing device described herein. Step 325 may be implemented in accordance with FIGS. 1 and 2 without limitation. It should be noted that, upon reading this disclosure, one or ordinary skill in the art would understand that the steps shown in FIG. 3 may be performed in any order in accordance with FIGS. 1 and 2 without limitation.

Still referring to FIG. 3, in some embodiments, a method may include administering to a subject in need thereof a composition comprising a glycosylation precursor, a modulator, and a delivery vehicle. In some embodiments, a modulator may include a P2Y12 inhibitor. In some embodiments, a delivery vehicle may include a prodrug delivery system. In some embodiments, a method may further include identifying a subject with insufficient functional V-ATPase. In some embodiments, a subject has a genetic mutation in a V-ATPase subunit gene. In some embodiments, a subject has a genetic mutation in ATP6AP2. In some embodiments, a glycosylation precursor comprises UDP-GLcNR (N-acetylglucosamine). In some embodiments, a glycosylation precursor may include ManNR (N-acetylmannosamine). In some embodiments, a glycosylation precursor may include an item selected from the list consisting of ManNR, ManNAc, UDP-GlcNac, UDP-GlcNR, ManNac-6-P, and SiaNR. In some embodiments, a prodrug delivery system may include a prodrug, wherein the prodrug of the prodrug delivery system is physiologically activated. In some embodiments, a prodrug delivery system may include a prodrug, wherein the prodrug of the prodrug delivery system is externally activated. In some embodiments, a prodrug delivery system may include a retrosynthetic design. In some embodiments, a prodrug delivery system may be capable of penetrating the blood brain barrier.

Now referring to FIG. 4, an illustrative example of a lysosomal process 400 is shown. Autophagy lysosomal pathway (ALP) is a major mechanism for degrading macromolecules. As used in this disclosure, “autophagy” is a conserved degradation of the cell that removes unnecessary or dysfunctional components through a lysosome-dependent regulated mechanism. Autophagy is the trafficking and waste management system found in nearly all cell types. Autophagy regulates and is regulated by a continuously expanding list of pathways in the body. The autophagosome 404 may be formed by numerous scaffolds and trafficking proteins and phosphorylation factors to eventually engulf damaged cargo such as improper proteins. Autophagy machinery may distinguish between dysfunctional and healthy cellular components and may initiate biogenesis of an autophagosome 404 around the dysfunctional components (i.e. pre-autophagic structure 412). Signaling pathways send proteins to chaperone scaffold proteins 408 that bind to the pre-autophagic structure 412. As a non-limiting example, pre-autophagic structure 412 may include cellular components as small as single proteins or as large and complex as mitochondria, protein aggregates, and pathogens. This cascade of binding and interaction may initiate the elongation, nucleation, maturation to form the autophagosome 404. The final step of autophagy may include the degradation process, which is initiated by the fusion of the autophagosome to the lysosome 416 creating the autolysosome 420.

The fusion of the lysosome 416 to the autophagosome 404 may deliver a specific pH and critical enzymes to complete degradation. As the final step of autophagy, the lysosome function may be an essential factor off waste management in cells in multiple organs. By-products of degradation can be recycled back into the cell. Deficiencies in lysosomal pH would disrupt autophagy and create a buildup of undegraded waste, which would be toxic to the cell. Defects in autophagy has been linked to many age-related diseases such as Parkinson's, Alzheimer's, frontotemporal Dementia, and Huntington's disease.

Still referring to FIG. 4, in addition to membrane repair, metabolic building-block sequestration, waste sequestration, and transcriptional regulation, the lysosome 416 degrades nearly all harmful waste in our cells. The pH of the lysosome 416 is measured below 5 whereas the cytosol is measured around 7.2. Lysosome ability to degrade waste and fuse with the autophagosome 404 is highly dependent on its low pH or high acidity and a number of enzymes.

Continuing to refer to FIG. 4, lysosomes 416 are the terminal step in multiple cellular pathways including the TFEB lysosomal signaling pathway. Lysosome health is also a regulator of upstream autophagy as part of a feedback pathway by providing building blocks of macromolecules. Waste-buildup that leads to cell death is seen in a vast number of diseases including cancers, neurodegeneration, cardiovascular disease, infectious disease, and aging.

Still referring to FIG. 4, PKA and cAMP are regulating pathways of lysosomes 416. P2Y12 inhibitors (antiplatelets) including but not limited to Clopidogrel (Plavix®), Prasugrel (Effient®), Ticlopidine (Ticlid®), Ticagrelor (Brilinta®), or the like may be used to activate the CAMP and PKA pathways by forcing the phosphorylation of the V-ATPase and restoring lysosomal acidification. Phosphorylating the V₀ subunit mimics the interaction of ATP6AP2 and ATP6AP1 as assembly factor of the V-ATPase. This restorative mechanism alleviates some of the autophagic symptoms as described herein.

Now referring to FIG. 5, an exemplary view of pH environments within a cell 500 is shown. cellular acidity (pH) is determined by H+ (cation/proton) concentrations and affects metabolism, membrane potential, cell growth, membrane transport, muscle function and much more. Organelles such as lysosomes 504, Golgi apparatus 508, and endoplasmic reticulum 512 maintain a specific organellular pH to create, modify, and degrade vital proteins. Glycosylation at the Golgi 508, lysosomal degradation, and autophagy are three critical pathways that are drastically impacted by a mutation of the V-ATPase assembly factors. As used in this disclosure, “glycosylation” is a process by which a carbohydrate is covalently attached to a target macromolecule, including for example, proteins and lipids.

Still referring to FIG. 5, Golgi 508 and endoplasmic reticulum 512 synthesize and package proteins. The pH at the ER 512 and Golgi 508 are precisely maintained for enzymes to carry out protein synthesis. Sialyltransferases are enzymes that attach sialic acids to proteins in glycosylation under specific pH conditions. Glycosylation is the post-translational (final) modification of proteins, which stabilizes the proteins and specifies them for a particular and necessary function. Glycosylation results in properly synthesized proteins, which contain sialic acids components. In a mutated V-ATPase, glycosylation is impaired, and proteins do not gain their functional unit and eventually misfold, creating harmful waste inside the cells.

Continuing to refer to FIG. 5, in a compromised pH environment, sialic acid binding is disrupted. Restoration may be achieved to a degree by adding one or more compounds that act as glycosylation precursors including but not limited to (i.e.: UDP-GlcNR (N-acetylglucosamine) and/or ManNR (N-acetylmannosamine). This potential therapeutic may improve all protein synthesis and modification in the Golgi and endoplasmic reticulum.

Now referring to FIGS. 6A-D, an exemplary embodiment of a structure 600 of a prodrug is illustrated. A common problem with drug delivery is low penetrance. The prodrug strategy has enhanced pharmacokinetic properties in a variety of applications including chemotherapy. The method of use application for this prodrug carrier increases efficacy of the administered dosage.

Prodrugs can program specificity to target tissues or cellular environments. Reducing off-target effects is widely sought after in drug safety. Prodrug delivery system may aid in crossing the blood brain barrier. In a non-limiting example, prodrug may include carrier prodrugs as illustrated in FIG. 6A. As a non-limiting example, carrier prodrug may include drug, linker and carrier. For example, and without limitation, carrier prodrugs may include folic acid, RGD peptide, sugars, or the like. In some embodiments, carrier prodrug may be physiologically activated. In another non-limiting example, prodrug may include decaging prodrug as shown in FIG. 6B. In some embodiments, decaging prodrug may be externally activated. In another non-limiting example, prodrug may include bioprecursor prodrug as shown in FIG. 6C. In some embodiments, bioprecursor prodrug may be physiologically activated. In another non-limiting example, prodrug may include synthetic prodrug as shown in FIG. 6D. In some embodiments, prodrug may be designed by finding a suitable reaction; for instance, metathesis, aromatization, or the like. Then, structure of prodrug, in a non-limiting example, may be optimized; for instance, by increasing cascade reactivity using scaffold choice and leaving group, increasing activity with biocatalyst using hydrophobic ester, increasing hydrolytic stability using pivalate ester, decreasing prodrug effect using bulky ester, or the like.

Still referring to FIGS. 6A-D, a unique front end and back end therapeutic approach may be implemented by the prodrug strategy. In particular, a front-end approach may include supplementation of N-acetylmannosamine may improve glycosylation effects on the liver. In addition, glycosylation effects in protein synthesis, protein translation and glycoprotein interaction with various organelles in liver, kidney, heart, brain, and spinal cord may be built upon. Further, the back-end approach may include an addition of a P2Y12, antiplatelet, may improve pH in retinal cell lysosomes, improve Golgi, ER, and lysosomal pH in multiple tissues the tissues previously listed.

Now referring to FIG. 7, a diagram of an exemplary prodrug delivery system 700 with ManNAc is shown. Prodrug delivery system 700 may be consistent with any prodrug delivery system 700 described in this disclosure. In some embodiments, prodrug asset may be included in a cocktail treatment of ATP6AP2 deficiency. Prodrug asset may be a ProTide prodrug which is just a category of prodrug that connects to a phosphate. Prodrug asset may be used with ManNAc-6-phosphate which is another glycosylation precursor. As shown in FIG. 7, a masked lipophilic ManNAc-6-P 704 may go through enzymatic activation 708 and enzymatic deacetylation 712 to yield a ManNAc-6-P 716 which is a precursor to sialic acid. In a non-limiting example, this may restore sialic acid in GNE-deficient cell lines at 1 mM. Glycosylation precursors may be used in treatment of ATP6AP2 as discussed further in this disclosure.

Now referring to FIG. 8, an illustration of an embodiment of the structure of V-ATPase is shown. In some embodiments, V₁ may be broadly responsible for hydrolyzing ATP and V₀ may be broadly responsible for transferring H+ across the membrane. V₁ may be made up of subunits A-H. Each V-ATPase complex may include 3 A subunits and 3 B subunits. ATP hydrolysis may occur at catalytic sites on the A and B subunits. The A and B subunits may also provide nucleotide binding sites for regulating V-ATPase activity. V₀ may be made up of subunits a, c, c″, d, e and Ac45 in mammals and a, c, c′, c″, d and e in yeast. V₁ and Vo may be connected by a central stalk made up of subunits D, F, and d as well as 3 peripheral stalks which are made up of subunits C, E, G, and part of subunit a. ATP hydrolysis may cause rotation of the central stalk and components of V₀ which form a ring. This rotation may cause components of this V₀ ring to pick up protons and deliver them to a channel on the other side of the membrane, where they are released.

Now referring to FIG. 9, an illustration of the function of V-ATPase (proton pump in FIG. 9) is provided. ATP is used to transport H+ from one side of a membrane to the other. This may be used by a cell to create an imbalance in pH across a membrane. The V-ATPase in this figure is on an exterior cell membrane, but V-ATPase may also be located on the membrane of various organelles such as Golgi apparatus and lysosomes.

Now referring to FIG. 10, an exemplary glycosylation process is illustrated. For example, Man I, GnT1, Man II, GnTII, and GnTV may perform steps in a glycosylation process. In some embodiments, glycosylation is pH dependent and may not occur properly when Golgi apparatus pH is abnormal due to a lack of functional V-ATPase.

Now referring to FIG. 11, an exemplary process of converting glycosylation precursors to sialic acid is illustrated. In some embodiments, addition of glycosylation precursors may improve glycosylation in contexts where Golgi apparatus pH is abnormal due to a lack of functional V-ATPase.

Now referring to FIG. 12, the Autophagy-Lysosomal Pathway as it relates to lysosomal fusion and degradation of waste is depicted. In some embodiments, a lack of functional V-ATPase may cause lysosome pH to be higher than normal. This may cause fusion of a lysosome to an autophagosome to fail to sufficiently reduce its pH. This may cause the autolysosome to fail to effectively degrade its contents.

Now referring to FIG. 13, TFEB, AMPK, and V-ATPase interaction as it relates to lysosome function and biogenesis is illustrated.

Now referring to FIG. 14, disclosed in FIG. 14 are chemical structures and pathways of one or more glycosylation precursors, including ManNR, UDP-GlcNR, and SiaNR.

Now referring to FIG. 15, metabolic effects of ATP6AP2 is illustrated. ATP6AP2 is known as the pro-renin receptor (PRP) for its role in regulating the renin-angiotensin system. This system plays an important role in regulating metabolic function including but not limited to kidney function, blood pressure, insulin sensitivity, lipid sensitivity, glucose metabolism, energy homeostasis, cell growth, cell survival, blood vessel generation, fibrosis, and inflammation. In some embodiments, V-ATPase malfunction may present as a metabolism malfunction. A “metabolism malfunction” as used in this disclosure is any disruption in a metabolic function of the human body. A metabolic function may include one or more biochemical reactions that occur within a living organism to sustain life. These biochemical reactions may enable cells to convert nutrients into energy, build and repair tissues, and regulate bodily functions. A metabolic function may include catabolismbreakdown processes such as converting molecules such as carbohydrates, fats and proteins into simpler ones to release energy. A metabolic function may include anabolism building processes such as utilization of energy to synthesize molecules such as proteins, nucleic acids, and lipids. A metabolic function may include energy production and utilization such as adenosine triphosphate (ATP) and mitochondria energy metabolism, including for example cellular respiration. A metabolic function may include metabolic regulation such as controlling enzymes, hormones, and cellular signals to balance energy needs. A metabolic malfunction may include any dysfunction of any metabolic function as described above. A metabolic malfunction may include but is not limited to inherited metabolic disorders such as lysosomal storage diseases (LSDs), amino acid metabolism disorders, carbohydrate metabolism disorders, fatty acid metabolism disorders and the like. A metabolic malfunction may include but is not limited to acquired metabolic disorders such as Type 1 Diabetes, Type 2 Diabetes, metabolic syndrome, obesity, lipid metabolism disorders, non-alcoholic fatty liver disease, hypothyroidism, hyperthyroidism, mitochondrial disorders and the like. A metabolic malfunction may include but is not limited to neurodegenerative diseases linked to metabolic dysfunction such as Alzheimer's disease, Parkinson's disease, Huntington's disease, and the like. A metabolic malfunction may include but is not limited to bone and connective tissue disorders such as osteoporosis, Ehlers-Danlos Syndrome (EDS) and the like. A metabolic malfunction may include a hormonal and genetic growth disorder such as Cushing's syndrome, growth hormone deficiency, and the like. Composition 200 and/or any combination as described herein may aid in resorting metabolic function through modulation of ATP6AP2 functionality and effects on pro-renin receptor via regulation of renin-angiotensin system. In some instances, metabolic effects may be modulated through restored function of mechanistic target of rapamycin (mTOR) as described in more detail below.

Now referring to FIG. 16, an illustration of V-ATPase malfunctions and mechanistic target of rapamycin (mTOR) signaling is illustrated. mTOR is a protein kinase that plays a role in regulating cell growth, metabolism, and autophagy. mTOR links with other proteins and serves as a core component of mTOR complex 1 and mTOR complex 2 which regulate various cell processes. For instance and without limitation, mTOR functions through a serine/threonine protein kinase to regulate cell growth, cell proliferation, cell motility, cell survival, protein synthesis, autophagy, and transcription. mTOR also functions through metabolic processes to promote activation of insulin receptors and insulin like growth factor receptors. Composition 200 and/or any combination as described herein aids in regenerating mTOR pathway signaling that may be disrupted as a function of a V-ATPase malfunction. Restoration of mTOR pathway signaling via administration of composition 200 and/or any combination as described herein has clinical applications for the treatment of aging, cancer, autism, Alzheimer's disease, lymphoproliferative diseases, protein synthesis and cell growth, scleroderma, and lysosomal damage that inhibits mTOR and induces autophagy. mTOR controls metabolic regulation through lysosomal catabolic activity. Under nutrient rich conditions when mTOR is active, lysosomes display an elevated pH and low protease activity. Additionally, a peripheral V-ATPase V1 domain resides in the cytosol along with TRIC. As mTOR activity declines, V1 domains move to the lysosome and assemble with V0 domains into functional proton pumps to acidy the organelle. Lysosomes serve as the cellular machinery that integrate nutrient availability and growth signals to regulate mTOR.

With continued reference to FIG. 16, composition 200 and/or any combination described herein causes VATPase to interact with SLC38A9 forming a scaffold to coordinate docking and function of mTOR signaling complex. This interaction directly connecting VATPase with SLC38A9 forming a scaffold aids in coordinating docking, binding, and function of mTOR signaling complex, ultimately promoting cell growth and regeneration. SLC38A9 (solute carrier family 38 member 9) is a lysosomal amino acid transporter and key regulator of the mechanistic target of rapamycin complex 1 (mTORC1) signaling pathway. It is a part of SLC38 family of amino acid transporters and is involved in sensing and transporting arginine and other amino acids from lysosomes to the cytoplasm. SLC38A9 plays a crucial role in activating mTORC1, a master regulator of cell growth and metabolism. In some instances, it senses lysosomal arginine levels and relays the information to mTORC1, which in turn regulates cellular metabolism, autophagy, and growth. SLC38A9 facilitates the efflux of essential amino acids, such as arginine, glutamine, and leucine from lysosomes to the cytosol, driving mTORC1 activation. SLC38A9 balances mTORC1 activation and inhibition. For example, during nutrient rich conditions, SLC38A9 promotes anabolic processes via activation of mTORC1. During nutrient depletion conditions, SLC38A9 suppresses mTORC1 activity, leading to autophagy. Ultimately, VATPase interaction with SLC38A9 fixes downstream consequences of poor assembly seen with V-ATPase malfunction.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A method of treating a V-ATPase malfunction, the method comprising:
identifying a malfunction of at least a portion of a V-ATPase protein in a subject;
manufacturing a composition comprising a glycosylation precursor, a modulator, and a delivery vehicle; and
administering the composition to the subject.

2. The method of claim 1, wherein the modulator comprises a P2Y12 inhibitor.

3. The method of claim 2, wherein the P2Y12 inhibitor is selected from the list consisting of clopidogrel, prasugrel, ticlopidine, and ticagrelor.

4. The method of claim 1, wherein the modulator comprises ticagrelor.

5. The method of claim 1, wherein the subject has a genetic mutation in a V-ATPase subunit gene.

6. The method of claim 5, wherein the subject has a genetic mutation in ATP6AP2.

7. The method of claim 1, wherein the glycosylation precursor comprises UDP-GLcNR (N-acetylglucosamine).

8. The method of claim 1, wherein the glycosylation precursor comprises ManNR (N-acetylmannosamine).

9. The method of claim 1, wherein the glycosylation precursor comprises a sialic acid precursor.

10. The method of claim 9, wherein the sialic acid precursor comprises an item selected from the list consisting of ManNR, ManNAc, UDP-GlcNac, UDP-GlcNR, ManNac-6-P, and SiaNR.

11. The method of claim 1, wherein the delivery vehicle comprises a prodrug delivery system.

12. The method of claim 11, wherein the prodrug delivery system comprises a prodrug, wherein the prodrug of the prodrug delivery system is physiologically activated.

13. The method of claim 11, wherein the prodrug delivery system comprises a prodrug, wherein the prodrug of the prodrug delivery system is externally activated.

14. The method of claim 11, wherein the prodrug delivery system comprises a retrosynthetic design.

15. The method of claim 11, wherein the prodrug delivery system is capable of facilitating delivery of the glycosylation precursor, the modulator, or both across the blood brain barrier.

16. The method of claim 1, wherein the composition increases glycosylation more than the glycosylation precursor alone.

17. The method of claim 1, wherein the composition improves lysosomal function more than the modulator alone.

18. The method of claim 1, wherein the composition activates MTOR pathway signaling disrupted as a function of the V-ATPase malfunction.

19. The method of claim 1, wherein the composition activates lysosomal biogenesis.

20. The method of claim 19, wherein lysosomal biogenesis is enhanced as a function of the transcription factor EB (TFEB) pathway.

21. The method of claim 1, wherein the V-ATPase malfunction further comprises a metabolism malfunction.

22. The method of claim 1, wherein the composition modulates calcium transport across a cell membrane.

23. The method of claim 1, wherein the composition enhances neuromodulation through APOE pathways.
